SEQUENCE LISTING (50 sequences)

SEQ ID NO: 1 - protein, 10 aa, Artificial Sequence
  VHH60 CDR1
GRTFRVNLMG

SEQ ID NO: 2 - protein, 11 aa, Artificial Sequence
  VHH60 CDR2
SINGFDDITYY

SEQ ID NO: 3 - protein, 15 aa, Artificial Sequence
  VHH60 CDR3
AYDSDYDGRLFNYWG

SEQ ID NO: 4 - protein, 10 aa, Artificial Sequence
  VHH35 CDR1
GSIYSFNFMG

SEQ ID NO: 5 - protein, 11 aa, Artificial Sequence
  VHH35 CDR2
TINSFDDITYY

SEQ ID NO: 6 - protein, 18 aa, Artificial Sequence
  VHH35 CDR3
VLGERTGISYGSAFDYWG

SEQ ID NO: 7 - protein, 10 aa, Artificial Sequence
  VHH79 CDR1
GFTSRNYFMG

SEQ ID NO: 8 - protein, 11 aa, Artificial Sequence
  VHH79 CDR2
TINSLSSITYY

SEQ ID NO: 9 - protein, 21 aa, Artificial Sequence
  VHH79 CDR3
VYTPTTGPGEGSYTPWHDYWG

SEQ ID NO: 10 - protein, 10 aa, Artificial Sequence
  VHH80 CDR1
GFISNFNLMG

SEQ ID NO: 11 - protein, 11 aa, Artificial Sequence
  VHH80 CDR2
TINSFDDITYY

SEQ ID NO: 12 - protein, 23 aa, Artificial Sequence
  VHH80 CDR3
AEVRSSLDYALWTSRRSAFSYWG

SEQ ID NO: 13 - protein, 10 aa, Artificial Sequence
  VHH34 CDR1
GFIYSFNIMG

SEQ ID NO: 14 - protein, 11 aa, Artificial Sequence
  VHH34 CDR2
SINWFSDITYY

SEQ ID NO: 15 - protein, 18 aa, Artificial Sequence
  VHH34 CDR3
AYLLRGDDRYYATYSYWG

SEQ ID NO: 16 - protein, 10 aa, Artificial Sequence
  VHH43 CDR1
GFISDADIMG

SEQ ID NO: 17 - protein, 11 aa, Artificial Sequence
  VHH43 CDR2
SINSYDSITYY

SEQ ID NO: 18 - protein, 12 aa, Artificial Sequence
  VHH43 CDR3
VRVHSRDFSYWG

SEQ ID NO: 19 - protein, 10 aa, Artificial Sequence
  VHH82 CDR1
GFIYSFNIMG

SEQ ID NO: 20 - protein, 11 aa, Artificial Sequence
  VHH82 CDR2
SISSYDDITYY

SEQ ID NO: 21 - protein, 18 aa, Artificial Sequence
  VHH82 CDR3
AYLLRGDDRYYATYSYWG

SEQ ID NO: 22 - protein, 121 aa, Artificial Sequence
  Amino acid sequence of VHH60
EVQLVESGGGLVQPGGSLRLSCAASGRTFRVNLMGWFRQAPGKGRELVASINGFDDITYYPDSVEGRFTISRDNAKRMVYLQMNSLRAEDTAVYYCAAYDSDYDGRLFNYWGQGTQVTVSS

SEQ ID NO: 23 - protein, 124 aa, Artificial Sequence
  Amino acid sequence of VHH35
EVQLVESGGGLVQPGGSLRLSCAASGSIYSFNFMGWFRQAPGKGRELVATINSFDDITYYPDSVEGRFTISRDNAKRMVYLQMNSLRAEDTAVYYCAVLGERTGISYGSAFDYWGQGTQVTVSS

SEQ ID NO: 24 - protein, 127 aa, Artificial Sequence
  Amino acid sequence of VHH79
EVQLVESGGGLVQPGGSLRLSCAASGFTSRNYFMGWFRQAPGKGRELVATINSLSSITYYPDSVEGRFTISRDNAKRMVYLQMNSLRAEDTAVYYCAVYTPTTGPGEGSYTPWHDYWGQGTQVTVSS

SEQ ID NO: 25 - protein, 129 aa, Artificial Sequence
  Amino acid sequence of VHH80
EVQLVESGGGLVQPGGSLRLSCAASGFISNFNLMGWFRQAPGKGRELVATINSFDDITYYPDSVEGRFTISRDNAKRMVYLQMNSLRAEDTAVYYCAAEVRSSLDYALWTSRRSAFSYWGQGTQVTVSS

SEQ ID NO: 26 - protein, 124 aa, Artificial Sequence
  Amino acid sequence of VHH34
EVQLVESGGGLVQPGGSLRLSCAASGFIYSFNIMGWFRQAPGKGRELVASINWFSDITYYPDSVEGRFTISRDNAKRMVYLQMNSLRAEDTAVYYCAAYLLRGDDRYYATYSYWGQGTQVTVSS

SEQ ID NO: 27 - protein, 118 aa, Artificial Sequence
  Amino acid sequence of VHH43
EVQLVESGGGLVQPGGSLRLSCAASGFISDADIMGWFRQAPGKGRELVASINSYDSITYYPDSVEGRFTISRDNAKRMVYLQMNSLRAEDTAVYYCAVRVHSRDFSYWGQGTQVTVSS

SEQ ID NO: 28 - protein, 124 aa, Artificial Sequence
  Amino acid sequence of VHH82
EVQLVESGGGLVQPGGSLRLSCAASGFIYSFNIMGWFRQAPGKGRELVASISSYDDITYYPDSVEGRFTISRDNAKRMVYLQMNSLRAEDTAVYYCAAYLLRGDDRYYATYSYWGQGTQVTVSS

SEQ ID NO: 29 - DNA, 363 bp, Artificial Sequence
  Nucleotide sequence encoding VHH60
gaggtgcagctggtggaaagcggcggaggactggtgcaacccggcggctctctgagactgagctgtgccgcctccggcagaacctttcgtgttaatcttatgggctggttcagacaagcccccggcaagggcagagagctggtggctagtattaacgggtttgatgatattacctattaccccgactccgtggagggaagattcaccatctctagagacaacgccaagaggatggtgtacctccagatgaactctctgagagccgaggacacagccgtgtattactgcgccgcttacgactctgactacgacggtcgtctgtttaattattggggacaaggcacccaagtgaccgtgagctcc

SEQ ID NO: 30 - DNA, 372 bp, Artificial Sequence
  Nucleotide sequence encoding VHH35
gaggtgcagctggtggaaagcggcggaggactggtgcaacccggcggctctctgagactgagctgtgccgcctccggcagtatctatagttttaattttatgggctggttcagacaagcccccggcaagggcagagagctggtggctactattaactcgtttgatgatattacctattaccccgactccgtggagggaagattcaccatctctagagacaacgccaagaggatggtgtacctccagatgaactctctgagagccgaggacacagccgtgtattactgcgccgttctgggtgaacgtactggtatctcttacggttctgcttttgattattggggacaaggcacccaagtgaccgtgagctcc

SEQ ID NO: 31 - DNA, 381 bp, Artificial Sequence
  Nucleotide sequence encoding VHH79
gaggtgcagctggtggaaagcggcggaggactggtgcaacccggcggctctctgagactgagctgtgccgcctccggctttacctctcgtaattattttatgggctggttcagacaagcccccggcaagggcagagagctggtggctactattaactcgcttagcagcattacctattaccccgactccgtggagggaagattcaccatctctagagacaacgccaagaggatggtgtacctccagatgaactctctgagagccgaggacacagccgtgtattactgcgccgtttacactccgactactggtccgggtgaaggttcttacactccgtggcatgactattggggacaaggcacccaagtgaccgtgagctcc

SEQ ID NO: 32 - DNA, 387 bp, Artificial Sequence
  Nucleotide sequence encoding VHH80
gaggtgcagctggtggaaagcggcggaggactggtgcaacccggcggctctctgagactgagctgtgccgcctccggctttatctctaactttaatcttatgggctggttcagacaagcccccggcaagggcagagagctggtggctactattaactcgtttgatgatattacctattaccccgactccgtggagggaagattcaccatctctagagacaacgccaagaggatggtgtacctccagatgaactctctgagagccgaggacacagccgtgtattactgcgccgctgaagttcgttcttctctggactacgctctgtggacttctcgtcgttctgcttttagttattggggacaaggcacccaagtgaccgtgagctcc

SEQ ID NO: 33 - DNA, 372 bp, Artificial Sequence
  Nucleotide sequence encoding VHH34
gaggtgcagctggtggaaagcggcggaggactggtgcaacccggcggctctctgagactgagctgtgccgcctccggctttatctatagttttaatattatgggctggttcagacaagcccccggcaagggcagagagctggtggctagtattaactggtttagcgatattacctattaccccgactccgtggagggaagattcaccatctctagagacaacgccaagaggatggtgtacctccagatgaactctctgagagccgaggacacagccgtgtattactgcgccgcttacctgctgcgtggtgacgaccgttactacgctacttatagctattggggacaaggcacccaagtgaccgtgagctcc

SEQ ID NO: 34 - DNA, 354 bp, Artificial Sequence
  Nucleotide sequence encoding VHH43
gaggtgcagctggtggaaagcggcggaggactggtgcaacccggcggctctctgagactgagctgtgccgcctccggctttatctctgacgctgatattatgggctggttcagacaagcccccggcaagggcagagagctggtggctagtattaactcgtatgatagcattacctattaccccgactccgtggagggaagattcaccatctctagagacaacgccaagaggatggtgtacctccagatgaactctctgagagccgaggacacagccgtgtattactgcgccgttcgtgttcattctcgtgactttagctattggggacaaggcacccaagtgaccgtgagctcc

SEQ ID NO: 35 - DNA, 372 bp, Artificial Sequence
  Nucleotide sequence encoding VHH82
gaggtgcagctggtggaaagcggcggaggactggtgcaacccggcggctctctgagactgagctgtgccgcctccggctttatctatagttttaatattatgggctggttcagacaagcccccggcaagggcagagagctggtggctagtattagctcgtatgatgatattacctattaccccgactccgtggagggaagattcaccatctctagagacaacgccaagaggatggtgtacctccagatgaactctctgagagccgaggacacagccgtgtattactgcgccgcttacctgctgcgtggtgacgaccgttactacgctacttatagctattggggacaaggcacccaagtgaccgtgagctcc

SEQ ID NO: 36 - protein, 15 aa, Artificial Sequence
  Linker
GSSSSGSSSSGSSSS

SEQ ID NO: 37 - protein, 19 aa, Artificial Sequence
  Signal peptide
MEFGLSWVFLVALFRGVQC

SEQ ID NO: 38 - protein, 12 aa, Artificial Sequence
  Linker
GSGGGGSGGGGS

SEQ ID NO: 39 - DNA, 22 bp, Artificial Sequence
  SARS2-N-F
taatcagacaaggaactgatta

SEQ ID NO: 40 - DNA, 19 bp, Artificial Sequence
  SARS2-N-R
cgaaggtgtgacttccatg

SEQ ID NO: 41 - DNA, 20 bp, Artificial Sequence
  SARS2-N-P
gcaaattgtgcaatttgcgg

SEQ ID NO: 42 - DNA, 20 bp, Artificial Sequence
  hGAPDH-F
cagcctcaagatcatcagca

SEQ ID NO: 43 - DNA, 20 bp, Artificial Sequence
  hGAPDH-R
tgtggtcatgagtccttcca

SEQ ID NO: 44 - DNA, 20 bp, Artificial Sequence
  hGAPDH-P
ctgcttagcacccctggcca

SEQ ID NO: 45 - DNA, 23 bp, Artificial Sequence
  hACE2-F
cattggagcaagtgttggatctt

SEQ ID NO: 46 - DNA, 22 bp, Artificial Sequence
  hACE2-R
gagctaatgcatgccattctca

SEQ ID NO: 47 - DNA, 26 bp, Artificial Sequence
  hACE2-P
cttgcagctacaccagttcccaggca

SEQ ID NO: 48 - DNA, 19 bp, Artificial Sequence
  mGAPDH-F
tgcaccaccaactgcttag

SEQ ID NO: 49 - DNA, 19 bp, Artificial Sequence
  mGAPDH-R
ggatgcagggatgatgttc

SEQ ID NO: 50 - DNA, 23 bp, Artificial Sequence
  mGAPDH-P
cagaagactgtggatggcccctc

(19) United States
(12) Patent Application Publication
(10) Pub. No.: US 2024/0158477 A1
(43) Pub. Date: May 16, 2024

(54) ANTIBODY AGAINST SARS-COV-2

(71) Applicant: Bioduro (Jiangsu) Co., Ltd., Wuxi (CN)

(72) Inventors: Xiang Li, Wuxi (CN); Yi Xiong, Wuxi (CN); Shin-Chen Hou, Wuxi (CN); Chenguang Cai, Wuxi (CN); Yanbin Guan, Wuxi (CN)

(73) Assignee: Bioduro (Jiangsu) Co., Ltd., Wuxi (CN)

(21) Appl. No.: 17/756,772

(22) PCT Filed: Jan. 25, 2022
(86) PCT No.: PCT/CN2022/073733

§ 371 Date: Jun. 2, 2022

(30) Foreign Application Priority Data
Jan. 27, 2021 (WO) PCT/CN2021/073917

(51) Int. Cl. C07K 16/10; A61K 45/06; A61K 47/68; A61P 31/14; A61K 39/00

(52) U.S. Cl. CPC C07K 16/1003; A61K 45/06; A61K 47/6841; A61P 31/14; A61K 2039/505

(57) ABSTRACT

A nanobody capable of specifically recognizing SARS-CoV-2 spike glycoprotein RBD is provided, and the nanobody comprises a CDR having an amino acid sequence selected from at least one of the following or at least 95% identical to the following: a CDR sequence of a heavy chain variable region: SEQ ID NO: 1˜21.

This international patent application claims the benefit of PCT Application No.: PCT/CN2021/073917 filed on Jan. 27, 2021, the entire content of which is incorporated by reference for all purpose.

### FIELD

The present invention relates to biotechnology, especially to a nanobody, an antibody, a nucleic acid molecule, an expression vector, a recombinant cell, an antibody-drug conjugate, a pharmaceutical composition, and uses thereof.

### BACKGROUND

The outbreak of coronavirus disease 2019 (COVID19) caused by a novel Severe Acute Respiratory Syndrome Coronavirus 2 (SARS-CoV-2) has last for one year and brought deleterious consequence to international economic and community activities, resulting in more than 90 million morbidities including 2 million mortalities worldwide, and the greatest economic collapse in this century. Infected Individuals underwent an estimated median viral incubation period of 7.76 days before showing clinical symptoms at 11.5 days post infection [1, 2]. Moreover, presymptomatic and around 15.6% asymptomatic patients contribute more than 40% cases [3, 4]. These epidemic characteristics have drastically limited epidemic prevention and control, and it's urgent to develop efficacious drugs to prevent virus transmission and offer antiviral therapies for infected patients. Nevertheless, the only drug approved for clinical use is Remdesivir, which was approved by Food and Drug Administration (FDA) for hospitalized patients which only covers 15% of all cases [5, 6].

SARS-CoV-2 is a single strand RNA virus, belongs to the genus Betacoronavirus, and shares genomic identity of 79.6% to SARS-CoV and 96.2% to a bat coronavirus RaTG13 [7-10]. Similar to other betacoronaviruses, SARS-CoV-2 infection is mediated by a glycoprotein Spike(S) binding to its receptor human angiotensin converting enzyme 2 (hACE2). S protein is a trimeric fusion protein on the virion surface, which is cleaved into receptor-binding fragment S1 and fusion fragment S2 by cellular serine protease TMPRSS2 and lysosomal proteases cathepsins upon engaging with host cell [11, 12]. S1 interacts with hACE2 by the Receptor Binding Domain (RBD) on its C-terminus, then switches conformation from “sitting-down” to “standing-up” to dissociate and expose S2 which drives virus fusion with cell membrane. Although sharing the same receptor as SARS-CoV, SARS-CoV-2 S protein demonstrates stronger affinity with hACE2 due to different amino acids of the S1/S2 cleavage site [12, 16], which partially explains the higher transmissibility. Remarkably SARS-CoV-2 not only attacks lung epithelia cell in airway but also other cell types like enterocytes, pancreatic beta cells due to the ubiquitously expression of hACE2 and TMPRSS2 [20-24]. The complexity of infection tropism significantly leads to severity and sequela of the disease, and therefore blocking virus infection in the first place is curial for disease control.

Currently several vaccines have been authorized for emergency use, and a number of vaccines and antibodies are under clinical trials at different phases [25]. Among all antibodies derived from human or small laboratory animals, another set of antibodies named nanobody demonstrates distinctive properties [32-34]. Nanobody (Nb), also called VHH, is single domain antibody fragment originally derived from camelids. Unlike conventional IgG antibody which comprises two light chains and heavy chains, nanobody only represents the monomeric target recognition module of the heavy chain while retains similar specificity and affinity. Given the small size of 15 kD, nanobody is easy to produce and manipulate, featuring robust thermostability, solubility and permeability, and low immunogenicity [35].

Recently, a nanobody based drug has been successfully approved by FDA for clinical use, validating the druggability of nanobodies as a special class of therapeutic antibodies [36].

### SUMMARY

The present invention is partially based on the following findings of the inventors.

The inventors screened a series of nanobodies from a phage displayed synthetic nanobody library, which are capable of binding to the Receptor Binding Domain (RBD) of SARS-CoV-2 spike glycoprotein(S) at single digit nanomolar concentration, protecting host cells from the viral infection.

Accordingly, in one aspect of present disclosure, an antibody binding to the SARS-CoV-2 spike glycoprotein or an antigen-binding fragment thereof is provided. In some embodiments, the antibody comprises a heavy chain variable region (VH) comprising one or more CDR(s) having an amino acid sequence selected from SEQ ID NOs: 1-21 or at least 90% identical to SEQ ID NOs: 1-21.

(SEQ ID NO: 1)
GRTFRVNLMG.
(SEQ ID NO: 2)
SINGFDDITYY.
(SEQ ID NO: 3)
AYDSDYDGRLFNYWG.
(SEQ ID NO: 4)
GSIYSFNFMG.
(SEQ ID NO: 5)
TINSFDDITYY.
(SEQ ID NO: 6)
VLGERTGISYGSAFDYWG.
(SEQ ID NO: 7)
GFTSRNYFMG.
(SEQ ID NO: 8)
TINSLSSITYY.
(SEQ ID NO: 9)
VYTPTTGPGEGSYTPWHDYWG.
(SEQ ID NO: 10)
GFISNFNLMG.
(SEQ ID NO: 11)
TINSFDDITYY.
(SEQ ID NO: 12)
AEVRSSLDYALWTSRRSAFSYWG.
(SEQ ID NO: 13)
GFIYSFNIMG.
(SEQ ID NO: 14)
SINWFSDITYY.
(SEQ ID NO: 15)
AYLLRGDDRYYATYSYWG.
(SEQ ID NO: 16)
GFISDADIMG.
(SEQ ID NO: 17)
SINSYDSITYY.
(SEQ ID NO: 18)
VRVHSRDFSYWG.
(SEQ ID NO: 19)
GFIYSFNIMG.
(SEQ ID NO: 20)
SISSYDDITYY.
(SEQ ID NO: 21)
AYLLRGDDRYYATYSYWG

The antibody according to the embodiment of the invention can specifically target and bind to SARS-CoV-2 spike glycoprotein RBD, inhibiting the binding of SARS-CoV-2 spike glycoprotein receptor to human angiotensin converting enzyme 2 (hACE2). The antibody according to the embodiment of the invention is a potential candidate for detecting SARS-CoV-2 and/or disease control against coronavirus disease 2019 (COVID-19).

In some embodiments of present disclosure, the above mentioned antibody may possess at least one of the following additional features:

In some embodiments of present disclosure, the VH comprises: a CDR1 having an amino acid sequence shown in any of SEQ ID NOs: 1, 4, 7, 10, 13, 16 and 19 or at least 90% identical to any one of SEQ ID NO: 1, 4, 7, 10, 13, 16 and 19; a CDR2 having an amino acid sequence shown in any of SEQ ID NOs: 2, 5, 8, 11, 14, 17 and 20 or at least 90% identical to any one of SEQ ID NO: 2, 5, 8, 11, 14, 17 and 20; and a CDR3 having an amino acid sequence shown in any of SEQ ID NOs: 3, 6, 9, 12, 15, 18 and 21 or at least 90% identical to any one of SEQ ID NO: 3, 6, 9, 12, 15, 18 and 21.

In some embodiments of present disclosure, the VH comprises a CDR1, a CDR2 and a CDR3 respectively having the amino acid sequences shown in SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, or SEQ ID NOs: 19-21.

In some embodiments of present disclosure, the antibody is univalent, bivalent or multivalent.

In some embodiments of present disclosure, the antibody is mono-specific, bi-specific or multi-specific.

In another aspect of present disclosure, a nanobody binding to SARS-CoV-2 spike glycoprotein is provided. In some embodiments of present disclosure, the nanobody comprises one or more CDR(s) having an amino acid sequence selected from SEQ ID NO: 1-21 or at least 90% identical to any one of SEQ ID NO: 1-21.

The nanobody according to the embodiment of the invention can specifically target and bind to SARS-CoV-2 spike glycoprotein RBD, inhibiting the binding of SARS-CoV-2 spike glycoprotein receptor to human angiotensin converting enzyme 2 (hACE2). The nanobody according to the embodiment of the invention is a potential candidate for detecting SARS-CoV-2 and/or disease control against coronavirus disease 2019 (COVID-19).

In some embodiments of present disclosure, the above mentioned nanobody may possess at least one of the following additional features:

In some embodiments of present disclosure, the nanobody comprises: a CDR1 having the sequence shown in any one of SEQ ID NO: 1, 4, 7, 10, 13, 16 and 19; a CDR2 having the sequence shown in any one of SEQ ID NO: 2, 5, 8, 11, 14, 17 and 20; and a CDR3 having the sequence shown in any one of SEQ ID NO: 3, 6, 9, 12, 15, 18 and 21.

In some embodiments of present disclosure, the nanobody comprises a CDR1, a CDR2 and a CDR3 respectively having the amino acid sequences shown in SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, or SEQ ID NOs: 19-21.

In some embodiments of present disclosure, the nanobody comprises a heavy chain frame region, and at least a part of the heavy chain frame region is derived from at least one of mouse antibody, human antibody, primate antibody and mutant thereof. Preferably, when the heavy chain frame region is derived from human antibody, the immunogenicity of the nanobody is lower.

In some embodiments of present disclosure, the nanobody has an amino acid sequence shown in any one of SEQ ID NO: 22-28.

(SEQ ID NO: 22)
EVQLVESGGGLVQPGGSLRLSCAASGRTFRVNLMGWFRQAPGKGRELVA
SINGFDDITYYPDSVEGRFTISRDNAKRMVYLQMNSLRAEDTAVYYCAA
YDSDYDGRLFNYWGQGTQVTVSS.
(SEQ ID NO: 23)
EVQLVESGGGLVQPGGSLRLSCAASGSIYSFNFMGWFRQAPGKGRELVA
TINSFDDITYYPDSVEGRFTISRDNAKRMVYLQMNSLRAEDTAVYYCAV
LGERTGISYGSAFDYWGQGTQVTVSS.
(SEQ ID NO: 24)
EVQLVESGGGLVQPGGSLRLSCAASGFTSRNYFMGWFRQAPGKGRELVA
TINSLSSITYYPDSVEGRFTISRDNAKRMVYLQMNSLRAEDTAVYYCAV
YTPTTGPGEGSYTPWHDYWGQGTQVTVSS.
(SEQ ID NO: 25)
EVQLVESGGGLVQPGGSLRLSCAASGFISNFNLMGWFRQAPGKGRELVA
TINSFDDITYYPDSVEGRFTISRDNAKRMVYLQMNSLRAEDTAVYYCAA
EVRSSLDYALWTSRRSAFSYWGQGTQVTVSS.
(SEQ ID NO: 26)
EVQLVESGGGLVQPGGSLRLSCAASGFIYSFNIMGWFRQAPGKGRELVA
SINWFSDITYYPDSVEGRFTISRDNAKRMVYLQMNSLRAEDTAVYYCAA
YLLRGDDRYYATYSYWGQGTQVTVSS.
(SEQ ID NO: 27)
EVQLVESGGGLVQPGGSLRLSCAASGFISDADIMGWFRQAPGKGRELVA
SINSYDSITYYPDSVEGRFTISRDNAKRMVYLQMNSLRAEDTAVYYCAV
RVHSRDFSYWGQGTQVTVSS.
(SEQ ID NO: 28)
EVQLVESGGGLVQPGGSLRLSCAASGFIYSFNIMGWFRQAPGKGRELVA
SISSYDDITYYPDSVEGRFTISRDNAKRMVYLQMNSLRAEDTAVYYCAA
YLLRGDDRYYATYSYWGQGTQVTVSS

The nanobody having the amino acid sequence shown in SEQ ID NO: 22 corresponds to clone VHH60 in the present disclosure; the nanobody having the amino acid sequence shown in SEQ ID NO:23 corresponds to clone VHH35; the nanobody having the amino acid sequence shown in SEQ ID NO:24 corresponds to clone VHH79; the nanobody having the amino acid sequence shown in SEQ ID NO:25 corresponding to clone VHH80, the nanobody shown in SEQ ID NO:26 is called VHH34 in this application, the nanobody shown in SEQ ID NO:27 is called VHH43 in this application, the nanobody shown in SEQ ID NO:28 is called VHH82 in this application.

In another aspect of present disclosure, a nucleic acid molecule encoding the nanobody or the antibody described above is provided. In some embodiments of present disclosure, the nucleic acid molecule may be introduced into a host cell to express the nanobody or the antibody described above.

In some embodiments of present disclosure, the above mentioned nucleic acid molecule may possess at least one of the following additional features:

In some embodiments of present disclosure, the nucleic acid molecule is DNA.

In some embodiments of present disclosure, the nucleic acid molecule has a nucleotide sequence shown in any one of SEQ ID NO: 29-35.

(SEQ ID NO: 29)
GAGGTGCAGCTGGTGGAAAGCGGCGGAGGACTGGTGCAACCCGGCGGCT
CTCTGAGACTGAGCTGTGCCGCCTCCGGCAGAACCTTTCGTGTTAATCT
TATGGGCTGGTTCAGACAAGCCCCCGGCAAGGGCAGAGAGCTGGTGGCT
AGTATTAACGGGTTTGATGATATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGCT
TACGACTCTGACTACGACGGTCGTCTGTTTAATTATTGGGGACAAGGCA
CCCAAGTGACCGTGAGCTCC.
(SEQ ID NO: 30)
GAGGTGCAGCTGGTGGAAAGCGGCGGAGGACTGGTGCAACCCGGCGGCT
CTCTGAGACTGAGCTGTGCCGCCTCCGGCAGTATCTATAGTTTTAATTT
TATGGGCTGGTTCAGACAAGCCCCCGGCAAGGGCAGAGAGCTGGTGGCT
ACTATTAACTCGTTTGATGATATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGTT
CTGGGTGAACGTACTGGTATCTCTTACGGTTCTGCTTTTGATTATTGGG
GACAAGGCACCCAAGTGACCGTGAGCTCC.
(SEQ ID NO: 31)
GAGGTGCAGCTGGTGGAAAGCGGCGGAGGACTGGTGCAACCCGGCGGCT
CTCTGAGACTGAGCTGTGCCGCCTCCGGCTTTACCTCTCGTAATTATTT
TATGGGCTGGTTCAGACAAGCCCCCGGCAAGGGCAGAGAGCTGGTGGCT
ACTATTAACTCGCTTAGCAGCATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGTT
TACACTCCGACTACTGGTCCGGGTGAAGGTTCTTACACTCCGTGGCATG
ACTATTGGGGACAAGGCACCCAAGTGACCGTGAGCTCC.
(SEQ ID NO: 32)
GAGGTGCAGCTGGTGGAAAGCGGCGGAGGACTGGTGCAACCCGGCGGCT
CTCTGAGACTGAGCTGTGCCGCCTCCGGCTTTATCTCTAACTTTAATCT
TATGGGCTGGTTCAGACAAGCCCCCGGCAAGGGCAGAGAGCTGGTGGCT
ACTATTAACTCGTTTGATGATATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGCT
GAAGTTCGTTCTTCTCTGGACTACGCTCTGTGGACTTCTCGTCGTTCTG
CTTTTAGTTATTGGGGACAAGGCACCCAAGTGACCGTGAGCTCC.
(SEQ ID NO: 33)
GAGGTGCAGCTGGTGGAAAGCGGCGGAGGACTGGTGCAACCCGGCGGCT
CTCTGAGACTGAGCTGTGCCGCCTCCGGCTTTATCTATAGTTTTAATAT
TATGGGCTGGTTCAGACAAGCCCCCGGCAAGGGCAGAGAGCTGGTGGCT
AGTATTAACTGGTTTAGCGATATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGCT
TACCTGCTGCGTGGTGACGACCGTTACTACGCTACTTATAGCTATTGGG
GACAAGGCACCCAAGTGACCGTGAGCTCC.
(SEQ ID NO: 34)
GAGGTGCAGCTGGTGGAAAGCGGCGGAGGACTGGTGCAACCCGGCGGCT
CTCTGAGACTGAGCTGTGCCGCCTCCGGCTTTATCTCTGACGCTGATAT
TATGGGCTGGTTCAGACAAGCCCCCGGCAAGGGCAGAGAGCTGGTGGCT
AGTATTAACTCGTATGATAGCATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGTT
CGTGTTCATTCTCGTGACTTTAGCTATTGGGGACAAGGCACCCAAGTGA
CCGTGAGCTCC.
(SEQ ID NO: 35)
GAGGTGCAGCTGGTGGAAAGCGGCGGAGGACTGGTGCAACCCGGCGGCT
CTCTGAGACTGAGCTGTGCCGCCTCCGGCTTTATCTATAGTTTTAATAT
TATGGGCTGGTTCAGACAAGCCCCCGGCAAGGGCAGAGAGCTGGTGGCT
AGTATTAGCTCGTATGATGATATTACCTATTACCCCGACTCCGTGGAGG
GAAGATTCACCATCTCTAGAGACAACGCCAAGAGGATGGTGTACCTCCA
GATGAACTCTCTGAGAGCCGAGGACACAGCCGTGTATTACTGCGCCGCT
TACCTGCTGCGTGGTGACGACCGTTACTACGCTACTTATAGCTATTGGG
GACAAGGCACCCAAGTGACCGTGAGCTCC.

The sequence shown in SEQ ID NO: 29 encodes a nanobody corresponding to clone VHH60; the sequence shown in SEQ ID NO: 30 encodes a nanobody corresponding to clone VHH35; the sequence shown in SEQ ID NO: 31 encodes a nanobody corresponding to clone VHH79; the sequence shown in SEQ ID NO: 32 encodes a nanobody corresponding to clone VHH80; the sequence shown in SEQ ID NO: 33 encodes a nanobody corresponding to clone VHH34; the sequence shown in SEQ ID NO: 34 encodes a nanobody corresponding to clone VHH43; and the sequence shown in SEQ ID NO: 35 encodes a nanobody corresponding to clone VHH82.

In another aspect of present disclosure, an expression vector comprising the nucleic acid molecule is provided. As described above, the nucleic acid molecule encoding the nanobody or the antibody of the present disclosure. Therefore, the expression vector introduced into a host cell according to the embodiment of the invention can express the nanobody or the antibody under suitable conditions for protein expression.

In some embodiments of present disclosure, the above mentioned expression vector may possess at least one of the following additional features:

In some embodiments of present disclosure, the expression vector is an eukaryotic expression vector.

In another aspect of present disclosure, a recombinant cell comprising the nucleic acid molecule or the expression vector for expressing the antibody or nanobody described above is provided.

In some embodiments of present disclosure, the above mentioned recombinant cell may possess at least one of the following additional features:

In some embodiments of present disclosure, the recombinant cell is obtained by introducing the expression vector described above into the host cell.

In some embodiments of present disclosure, the recombinant cell is a eukaryotic cell.

In some embodiments of present disclosure, the recombinant cell is a mammalian cell, e.g., CHO.

In another aspect of present disclosure, an antibody-drug conjugate comprising the antibody or the nanobody described above conjugated to a therapeutic agent, a diagnostic agent or an imaging agent is provided.

In some embodiments of present disclosure, the antibody-drug conjugate comprises the nanobody or the antibody described, a linker and a therapeutic agent, a diagnostic agent or an imaging agent.

In some embodiments of present disclosure, the therapeutic agent is a small molecule cytotoxic drug.

The antibody-drug conjugate according to the embodiment of the invention can target and act on the virus under the guidance of nanobody or antibody targeting, so as to realize the targeted effect to detect the coronavirus 2 or inhibit the coronavirus 2.

In another aspect of present disclosure, a pharmaceutical composition comprising the nanobody, the antibody, the nucleic acid molecule, the expression vector, the recombinant cell, and/or the antibody-drug conjugate described above is provided. The pharmaceutical composition according to the embodiment of the invention is a potential candidate for detecting SARS-CoV-2, or preventing, treating or lessening of a disease caused by SARS-CoV-2 infection.

In some embodiments of present disclosure, the pharmaceutical composition further comprises a pharmaceutically acceptable carrier, excipient or diluent.

In another aspect of present disclosure, use of the nanobody, the antibody, the nucleic acid molecule, the expression vector, the recombinant cell, and the antibody-drug conjugate or the pharmaceutical composition described above in the manufacture of a medicament for preventing, treating or lessening of a disease caused by SARS-CoV-2 infection is provided.

In another aspect of present disclosure, use of the nanobody, the antibody, the nucleic acid molecule, the expression vector, the recombinant cell, and the antibody-drug conjugate or the pharmaceutical composition in the manufacture of a medicament for inhibiting binding of the spike glycoprotein of SARS-CoV-2 to human angiotensin converting enzyme 2 or blocking SARS-CoV-2 infection is provided.

In another aspect of present disclosure, the nanobody, the antibody, the nucleic acid molecule, the expression vector, the recombinant cell, and the antibody-drug conjugate or the pharmaceutical composition described above, for use in preventing, treating or lessening of a disease caused by SARS-CoV-2 infection is provided.

In another aspect of present disclosure, the nanobody, the antibody, the nucleic acid molecule, the expression vector, the recombinant cell, and the antibody-drug conjugate or the pharmaceutical composition, for use in inhibiting binding of the spike glycoprotein of SARS-CoV-2 to human angiotensin converting enzyme 2 or blocking SARS-CoV-2 infection is provided.

In another aspect of present disclosure, a method of preventing, treating or lessening of a disease caused by SARS-CoV-2 infection is provided. In some embodiments of present disclosure, the method comprises administering to the patient a therapeutically effective amount of the nanobody, the antibody, the nucleic acid molecule, the expression vector, the recombinant cell, and the antibody-drug conjugate or the pharmaceutical composition described above.

In another aspect of present disclosure, a method of inhibiting binding of the spike glycoprotein of SARS-CoV-2 to human angiotensin converting enzyme 2 or blocking SARS-CoV-2 infection is provided. In some embodiments of present disclosure, the method comprises giving the sample or administering to the subject an effective amount of the nanobody, the antibody, the nucleic acid molecule, the expression vector, the recombinant cell, and the antibody-drug conjugate or the pharmaceutical composition described above.

In another aspect of present disclosure, a kit for the detection of SARS-CoV-2 spike glycoprotein RBD or SARS-CoV-2 spike glycoprotein or SARS-CoV-2 is provided. In some embodiments of present disclosure, the kit comprises the nanobody, the antibody or the antibody-drug conjugate described above.

In another aspect of present disclosure, use of the nanobody, the antibody, the nucleic acid molecule, the expression vector, the recombinant cell, and the antibody-drug conjugate or the pharmaceutical composition described above in the manufacture of a kit for detecting SARS-CoV-2 spike glycoprotein RBD, SARS-CoV-2 spike glycoprotein or SARS-CoV-2 is provided.

In another aspect of present disclosure, the nanobody, the antibody, the nucleic acid molecule, the expression vector, the recombinant cell, and the antibody-drug conjugate or the pharmaceutical composition described above, for use in detecting SARS-CoV-2 spike glycoprotein RBD, SARS-CoV-2 spike glycoprotein or SARS-CoV-2 is provided.

In another aspect of present disclosure, a method of detecting SARS-CoV-2 spike glycoprotein RBD, SARS-CoV-2 spike glycoprotein or SARS-CoV-2 is provided. In some embodiments of present disclosure, the method comprises giving the nanobody, the antibody, the nucleic acid molecule, the expression vector, the recombinant cell, and the antibody-drug conjugate or the pharmaceutical composition described above to the sample to be tested.

More aspects and advantages will be described below, at least a part thereof will be clear in the following description accompanying the figures as attached, and/or be obvious for a person normally skilled in the art from embodiments described herein after.

### BRIEF DESCRIPTION OF THE FIGURES

The aforementioned features and advantages of the invention as well as additional features and advantages thereof will be more clearly understood hereafter as a result of a detailed description of the following embodiments when taken in conjunction with the drawings, wherein:

FIG. 1 shows the procedure of RBD binding nanobody screening. The phage displayed synthetic nanobody library was used for bio panning against immobilized Fc tagged RBD protein. After 3 rounds of panning, single clone phage ELISA was performed for the identification of RBD binding nanobodies. After sequencing, the unique clones were subjected to PCR rescue of VHH genes followed by an overlap PCR to assemble the promoter and Fc fragment with VHH as a VHH-Fc mammalian expression cassette. The PCR products were transfected into ExpiCHO cells for expression; the supernatants were used for downstream assay for the identification of nanobodies blocking the interaction of RBD with hACE2.

FIG. 2 shows the competition ELSIA assay for RBD blocking nanobodies. The culture medium of ExpiCHO cells with expressed VHH-Fc was used for competition ELSIA to screen for the nanobodies that block the binding of RBD to the coated hACE2. As controls, hACE2-Fc, and VHH72-Fc (PC VHH-Fc) showed inhibition of RBD binding to the coated hACE2 protein. In the experiment wells, the hACE2-Fc was replaced by each of the 78 RBD VHH clones. The blockage of RBD binding to hACE2 was measured by the reduction of OD450 signal generated by the RBD protein.

FIG. 3 shows the SDS-PAGE assay for purified Fc tagged nanobodies. The Fc tagged nanobodies were purified by protein A resin from the culture medium of ExpiCHO cells. 2 ug of proteins were used for SDS-PAGE analysis in reduced and non-reduced conditions.

FIG. 4 shows the ELISA test of Fc tagged nanobodies. The purified Fc tagged nanobodies were serial diluted and subjected to RBD coated immunoplate to test the affinity. Fc tagged VHH72 and hACE2 were used as a reference and a positive control respectively.

FIG. 5 shows the multi-concentration affinity measurement of Fc tagged nanobodies by SPR. The Fc tagged nanobodies were captured onto the Protein A Chip, and a RBD protein in a series of concentrations were used to measure the affinity (dilution ratio: 2; concentration levels: at least 5 (excluding curves with irregularities or high background); duplicate concentrations included). All data were double-referenced prior to fitting using the 1:1 kinetics binding model in Biacore Insight Evaluation Software v3.0, GE to determine apparent KD.

FIG. 6 shows the blocking of RBD/hACE2 interaction evaluated by SPR. The Fc tagged nanobodies and a reference antibody (Novoprotein Neutralizing Antibody) were captured onto the Protein A Chip as indicated at the first curve. The second binding curve was detected when 50 nM of RBD (COVID-19 S.P.RBD) was injected. Lastly, injection of 100 nM hACE2 (ACE2) showed no further binding curve in all the experiments.

FIG. 7 shows the neutralization activity of nanobodies tested with a pseudovirus. The inhibition by nanobodies of the infection of SARS-CoV-2 by the pseudovirus expressing SARS-CoV-2 S protein and luciferase was measured. The percentage relative luciferase activity reflecting virus infection to control was calculated and the curves were fitted to extract IC₅₀ values, which are shown in parentheses (in nM).

FIG. 8 shows neuralization activity of nanobodies tested by authentic SARS-CoV-2. The neuralization of infection of SARS-CoV-2 virus against Vero E6 cells mediated by nanobodies was measured by RNA levels in the Vero E6 cells. Fc tagged nanobody-mediated neutralization of virus expressed as percentage relative infection, and infection curves were fitted to extract IC50 values, which are shown in parentheses (in nM).

FIG. 9 shows VHH60 mediated protection of mice from lethal infection of SARS-COV-2. A, Scheme of animal challenge. Total 10 mice were separated in each group, 5 mice were sacrificed 3 d.p.i., and all remaining mice will be terminated after meeting certain criteria. B, Survival curve of mice infected by authentic SARS-CoV-2. Mice in vehicle group all died at 4 d.p.i (5/5), one in VHH60 group (1/5) died. C, Body weight change of mice infected by authentic SARS-CoV-2. Data is represented as ratio of body weights at indicated timepoint versus day 0 (n=10 at 0 and 3 d.p.i, n=4 at 4 d.p.i, n=5 at 5.d.p.i).

FIG. 10 shows VHH60 mediated reduction of the viral load in the lung of mice infected by SARS-COV-2. A, Virus load in lung after 3 days of infection (n=5). B, Representative image of immunofluorescence from lung. Blue: Nuclei, Red: ACE2, Green: nucleocapsid protein (NP). Upper panel: whole section, Lower panel: zoomed from white squares at upper panel (n=3). *:P<0.05

FIG. 11 shows VHH60 mediated blocking of escape mutants and prevailing variants. A, VHH60 inhibits pseudovirus carrying spike protein with single mutation from infecting CaCO2 cell line (n=2). B, VHH60 inhibits pseudovirus carrying spike protein with multiple mutations as in reported variants from infecting CaCO2 cell line.

### DETAILED DESCRIPTION OF THE EMBODIMENTS

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

Unless indicated otherwise, the term “immunoglobulin sequence”—whether it is used herein to refer to a heavy chain antibody or a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “Vim sequence” or “protein sequence”), should generally be understood as to include both the relevant amino acid sequence as well as nucleic acid sequences or nucleotide sequences encoding the same, unless the context requires a more limited interpretation.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein.

For the purposes of comparing two or more nucleotide sequences, the percentage of “sequence identity” between a first sequence and a second sequence may be calculated by dividing [the number of nucleotides in the first sequence that are identical to the nucleotides at the corresponding positions in the second sequence] by [the total number of nucleotides/amino acids in the first sequence] and multiplying by [100%], in which each deletion, insertion, substitution or addition of a nucleotide in the second nucleotide sequence—compared to the first nucleotide sequence—is considered as a difference at a single nucleotide (position).

Alternatively, the degree of sequence identity between two or more nucleotide sequences may be calculated using a known computer algorithm for sequence alignment such as NCBI Blast v2.0, using standard settings.

Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

For the purposes of comparing two or more amino acid sequences, the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of nucleotides in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.

Alternatively, the degree of sequence identity between two amino acid sequences may be calculated using a known computer algorithm, such as those mentioned above for determining the degree of sequence identity for nucleotide sequences, again using standard settings.

Usually, for the purpose of determining the percentage of “sequence identity” between two amino acid sequences in accordance with the calculation method outlined herein above, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nat. Acad Sci. USA 81: 140-144, 1984; Kyte & Doolittle, J Mol. Biol. 157: 105-132, 1981, and Goldman et al., Ann. Rev. Biophys. Chem. 15: 321-353, 1986, all incorporated herein in their entirety by reference. Information on the primary, secondary and tertiary structure of nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); Vol. 3, 752-757; and Decanniere et al., Structure, Vol. 7, 4, 361 (1999). Further information is given on some of the amino acid residues that in conventional V_{H} domains form the V_{H}/V_{L} interface and potential camelizing substitutions on these positions.

Amino acid sequences and nucleic acid sequences are said to be “identical” if they have 100% sequence identity (as defined herein) over their entire length.

A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis.

The term ‘antigenic determinant’ refers to the epitope on the antigen recognized by the antigen-binding molecule (such as a nanobody of the invention) and more in particular by the antigen-binding site of said molecule. The terms “antigenic determinant” and “epitope’ may also be used interchangeably herein.

An amino acid sequence (such as a nanobody, an antibody) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.

The term “specificity” refers to the number of different types of antigens or antigenic determinants to which a particular antigen-binding molecule or antigen-binding protein (such as a nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). Avidity is the measure of the strength of binding between an antigen-binding molecule (such as a nanobody of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the nanobodies and/or polypeptides of the invention) will bind with a dissociation constant (KD) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ mol/liter is generally considered to indicate non-specific binding. Preferably, a nanobody of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

As further described herein, the amino acid sequence and structure of a nanobody can be considered—without however being limited thereto—to be comprised of four framework regions or “FRs”, which are referred to in the art and herein as “Framework region 1” or “FR1”; as “Framework region 2” or“FR2”; as “Framework region 3” or “FR3”; and as “Framework region 4” or “FR4”, respectively; these framework regions are interrupted by three complementary determining regions or “CDRs”, which are referred to in the art as “Complementarity Determining Region 1’ or “CDR1”; as “Complementarity Determining Region 2” or “CDR2”; and as “Complementarity Determining Region 3” or “CDR3”, respectively.

As also further described herein, the total number of amino acid residues in a nanobody can be in the range of 120-130, is preferably 121-129, and is most preferably 121. It should however be noted that parts, fragments, analogs or derivatives (as further described herein) of a nanobody are not particularly limited as to their length and/or size, as long as such parts, fragments, analogs or derivatives meet the further requirements outlined herein and are also preferably suitable for the purposes described herein.

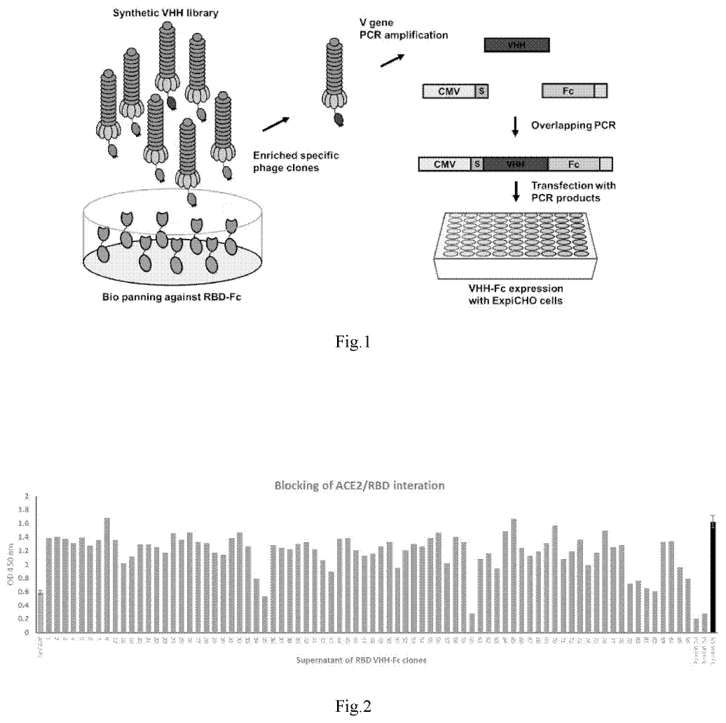

The amino acid residues of a nanobody are numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, referred to above (see for example FIG. 2 of said reference). In this respect, it should be noted that—as is well known in the art for V_{H} domains and for V_{HH} domains—the total number of amino acid residues in each of the CDRs may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of V_{H} domains, which can also be applied in an analogous manner to V_{HH} domains from Camelids and to nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise.

In accordance with the terminology used in the above references, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv fragments, which consist of a V_{H} domain covalently linked to a V_{L} domain).

Because of these unique properties, the use of V_{HH} domains and nanobodies as single antigen-binding proteins or as antigen-binding domains (i.e. as part of a larger protein or polypeptide) offers a number of significant advantages over the use of conventional V_{H} and V_{L} domains, ScFv or conventional antibody fragments (such as Fab- or F(ab′)2-fragments): only a single domain is required to bind an antigen with high affinity and with high selectivity, so that there is no need to have two separate domains present, nor to assure that these two domains are present in the right spatial conformation and configuration (i.e. through the use of specially designed linkers, as with ScFv's).

V_{HH} domains and nanobodies can be expressed from a single gene and require no post-translational folding or modifications.

V_{HH} domains and nanobodies can easily be engineered into multivalent and multispecific formats (as further discussed herein).

V_{HH} domains and nanobodies are highly soluble and do not have a tendency to aggregate (as with the mouse-derived antigen-binding domains” described by Ward et al., Nature, Vol. 341, 1989, p. 544).

V_{HH} domains and nanobodies are highly stable to heat, pH, proteases and other denaturing agents or conditions (see for example Ewert et al, supra).

V_{HH} domains and nanobodies are easy and relatively cheap to prepare, even on a scale required for production. For example, V_{HH} domains, nanobodies and proteins/polypeptides containing the same can be produced using microbial fermentation (e.g. as further described below) and do not require the use of mammalian expression systems, as with for example conventional antibody fragments.

V_{HH} domains and nanobodies are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof.

V_{HH} domains and nanobodies can show so-called cavity-binding properties (inter alia due to their extended CDR3 loop, compared to conventional V_{H} domains) and can therefore also access targets and epitopes not accessible to conventional 4-chain antibodies and antigen-binding fragments thereof. For example, it has been shown that V_{HH} domains and nanobodies can inhibit enzymes (see for example WO 97/49805; Transue et al., (1998), supra; and Lauwereys et al., (1998), supra).

As mentioned above, the invention generally relates to nanobodies directed against SARS-CoV-2 spike glycoprotein(S) Receptor Binding Domain (RBD), as well as to polypeptides comprising or essentially consisting of one or more of such nanobodies, that can be used for the prophylactic, therapeutic and/or diagnostic purposes described herein.

As also further described herein, the invention further relates to nucleic acids encoding such nanobodies, to methods for preparing such nanobodies, to host cells expressing or capable of expressing such nanobodies, to compositions comprising such nanobodies, nucleic acids or host cells, and to uses of such nanobodies, nucleic acids, host cells or compositions.

Generally, it should be noted that the term nanobody as used herein in its broadest sense is not limited to a specific biological source or to a specific method of preparation.

In a first preferred, but non-limiting aspect, a nanobody of the invention may have the structure

FR1-CDR1-FR2-CDR2-FR3-CDR3-FR4
in which FR1 to FR4 refer to framework regions 1 to 4, respectively, and in which CDR1 to CDR3 refer to the complementarity determining regions 1 to 3, respectively.

A humanized nanobody of the invention may be as defined herein, but with the proviso that it has at least “one amino acid difference” (as defined herein) in at least one of the framework regions compared to the corresponding framework region of a naturally occurring V_{HH} domain. More specifically, according to one non-limiting aspect of the invention, a nanobody may be as defined herein, but with the proviso that it has at least “one amino acid difference” (as defined herein) at least one of the Hallmark residues (including those at positions 108, 103 and/or 45) compared to the corresponding framework region of a naturally occurring V_{HH} domain. Usually, a nanobody will have at least one such amino acid difference with a naturally occurring V_{HH} domain in at least one of FR2 and/or FR4, and in particular at least one of the Hallmark residues in FR2 and/or FR4.

Another embodiment of the present invention is a nucleic acid capable of encoding a nanobody or antibody as defined above.

Another embodiment of the present invention is an antibody-drug conjugate comprising the nanobody or the antibody described, a linker and a small molecule cytotoxic drug. Antibody-drug conjugate (ADC) is a chemical link that connects a bioactive small molecule drug to an antibody, e.g., the nanobody or the antibody of the invention, which acts as a carrier to deliver the small molecule drug to target cells.

Another embodiment of the present invention is a composition comprising a nanobody and/or nucleic as defined above.

Another embodiment of the present invention is a composition as defined above further comprising a pharmaceutically acceptable vehicle.

Another embodiment of the present invention is as defined above, or a nucleic acid as defined above, or a composition as defined above for use as a medicament.

Another embodiment of the present invention is a polypeptide as defined above, or a nucleic acid as defined above, or a composition as defined above for use in the treatment, prevention and/or alleviation of disorders mediated by SARS-CoV-2 infection.

Another embodiment of the present invention is the use of a nanobody as defined above, or a nucleic acid as defined above, or a composition as defined above for the preparation of a medicament for the treatment, prevention and/or alleviation of disorders mediated by SARS-CoV-2 infections.

Another embodiment of the present invention is a nanobody, nucleic acid or composition or use thereof as defined above wherein said disorder is the coronavirus disease 2019 (COVID-19).

Another embodiment of the present invention is a nanobody, nucleic acid or composition as defined above or the use of a nanobody as defined above wherein said nanobody is administered intravenously, subcutaneously, orally, sublingually, nasally or by inhalation.

Another embodiment of the present invention is a method of prophylactically or therapeutically treating COVID-19, comprising administering to the patient an effective dosage of a composition as defined above.

Another embodiment of the present invention is a method of producing a nanobody as defined above comprising:
a) culturing host cells comprising nucleic acids capable of encoding a polypeptide as defined above under conditions allowing the expression of the polypeptide, and,
b) recovering the produced polypeptide from the culture.

Another embodiment of the present invention is a method as defined above, wherein said host cells are bacterial, yeast or mammalian cells.

Another embodiment of the present invention is a method of diagnosing a disease or disorder mediated by SARS-CoV-2 infection comprising the steps of:
a) contacting a sample with a nanobody as defined above, and
b) detecting binding of said nanobody to said sample, and
c) comparing the binding detected in step (b) with a standard, wherein a difference in binding relative to said sample is diagnostic of a disease or disorder characterized by SARS-CoV-2 infection.

Another embodiment of the present invention is a method of diagnosing a disease or disorder mediated by SARS-CoV-2 infection comprising the steps of:
a) contacting a sample with a nanobody as defined above, and
b) determining the amount of Spike glycoprotein(S) or Spike glycoprotein(S) RBD in the sample
c) comparing the amount determined in step (b) with a standard, wherein a difference in amount relative to said sample is diagnostic of a disease or disorder characterized by SARS-CoV-2 infection.

Another embodiment of the present invention is a kit for diagnosing a disease or disorder mediated by SARS-CoV-2 infection for use in a method as defined above.

Another embodiment of the present invention is a kit for detection of SARS-CoV-2 spike glycoprotein RBD or SARS-CoV-2 spike glycoprotein or SARS-CoV-2 for use in a method as defined above.

Another embodiment of the present invention is a nanobody as defined above further comprising one or more in vivo imaging agents.

One embodiment of the present invention relates to a pharmaceutical composition comprising at least one nanobody of the invention and at least a pharmaceutically acceptable carrier, diluent or excipients.

The anti-RBD nanobodies of the present invention bind to SARS-CoV-2 spike glycoprotein RBD. According to one aspect of the invention, the anti-RBD nanobody binds to a target A-beta, and inhibits its interaction with one or more other hACE2.

An ELISA assay to measure the binding of an anti-RBD nanobody is well known.

The anti-RBD polypeptides as disclosed herein and their derivatives not only possess the advantageous characteristics of conventional antibodies, such as low toxicity and high selectivity, but they also exhibit additional properties. They are more soluble; as such they may be stored and/or administered in higher concentrations compared with conventional antibodies.

Conventional antibodies are not stable at room temperature, and have to be refrigerated for preparation and storage, requiring necessary refrigerated laboratory equipment, storage and transport, which contribute towards time and expense. The anti-RBD nanobodies of the present invention are stable at room temperature; as such they may be prepared, stored and/or transported without the use of refrigeration equipment, conveying a cost, time and environmental savings. Furthermore, conventional antibodies are unsuitable for use in assays or kits performed at temperatures outside biologically active-temperature ranges (e.g. 37±20° C.).

Other advantageous characteristics of the anti-RBD nanobodies as disclosed herein as compared to conventional antibodies include modulation of half-life in the circulation which may be modulated according to the invention by, for example, albumin-coupling, or by coupling to one or more nanobodies directed against a serum protein such as, for example, serum albumin. One aspect of the invention is a bispecific anti-RBD nanobody, with one specificity against a serum protein such as serum albumin and the other against the target as disclosed in WO04/041865 and incorporated herein as a reference. Other means to enhance half-life include coupling a polypeptide of the present invention to Fc, or to other nanobodies directed against RBD (i.e. creating multivalent nanobodies—bivalent, trivalent, etc.) or coupling to polyethylene glycol. A controllable half-life is desirable for modulating dosage with immediate effect.

Conventional antibodies are unsuitable for use in environments outside the usual physiological pH range. They are unstable at low or high pH and hence are not suitable for oral administration. Camelidae antibodies resist harsh conditions, such as extreme pH, denaturing reagents and high temperatures, so making the anti-RBD antibodies as disclosed herein suitable for delivery by oral administration. Camelidae antibodies are resistant to the action of proteases which is less so for conventional antibodies.

The anti-A-beta polypeptides as disclosed herein are less immunogenic than conventional antibodies. A subclass of Camelidae antibodies has been discovered which displays 95% amino acid sequence homology to human V_{H} framework regions. This suggests that immunogenicity upon administration in human patients can be anticipated to be minor or even non-existent. Alternatively, if so required, humanization of nanobodies surprisingly requires only a few residues that need to be substituted.

One aspect of the invention is an anti-RBD polypeptide comprising at least one anti-RBD heavy chain antibody, and in particular a nanobody derived therefrom. It is an aspect of the invention that such a polypeptide may comprise additional components. Such components may be polypeptide sequences, for example, one or more anti-A-RBD nanobodies, one or more anti-serum albumin nanobodies. Other fusion proteins are within the scope of the invention, and may include, for example, fusions with carrier polypeptides, signaling molecules, tags, and enzymes. Other components may include, for example, radiolabels, organic dyes, fluorescent compounds.

According to an aspect of the invention, an anti-RBD polypeptide of the invention may comprise at least two identical or non-identical anti-RBD nanobody sequences. It may be an aspect of the invention that at least two of the aforementioned sequences do not have equal affinity for RBD, so forming an anti-RBD polypeptide combining weak and high affinity binding sequences.

Methods of constructing bivalent polypeptides are known in the art (e.g. US 2003/0088074), and are also described below.

It may be desirable to modify the anti-RBD polypeptide of the invention with respect to effector function so as to enhance its therapeutic efficacy. For example, nanobody-fusions with certain Fc domains may be advantageous, especially with Fc domains of human origin.

In sequential administration, a polypeptide may be administered once, or any number of times and in various doses before and/or after administration of the agent. Sequential administration may be combined with simultaneous or sequential administration.

Another embodiment of the present invention is an anti-RBD polypeptide as described herein in which one or more nanobodies is humanized. The humanized nanobody may be an anti-RBD nanobody, an anti-serum albumin, other nanobodies useful according to the invention, or a combination of these.

By humanized is meant mutated so that potential immunogenicity upon administration in human patients is minor or nonexistent. Humanizing a polypeptide, according to the present invention, may comprise a step of replacing one or more of the non-human immunoglobulin amino acids by their human counterparts as found in a human consensus sequence or human germline gene sequence, without that polypeptide losing its typical character, i.e. the humanization does not significantly affect the antigen binding capacity of the resulting polypeptide.

According to one aspect of the invention, a humanized nanobody is defined as a nanobody having at least 50% homology {e.g. 55%, 60%, 65%, 70%, 75%, 80%, 85%, 90%, 95%, 98%, 100%) to the human framework region.

The inventors have determined the amino acid residues of a nanobody which may be modified without diminishing the native affinity, in order to reduce its immunogenicity with respect to a heterologous species.

The inventors have also found that humanization of nanobody polypeptides requires the introduction and mutagenesis of only a limited number of amino acids in a single polypeptide chain without a dramatic loss of binding and/or inhibitory activity. This is in contrast to humanization of ScFv, Fab, (Fab′)2 and IgG, which requires the introduction of amino acid changes in two chains, the light and the heavy chain, and the preservation of the assembly of both chains.

A homologous sequence of the present invention may include an anti-RBD polypeptide which has been humanized. The humanization of antibodies of the new class of nanobodies would further reduce the possibility of unwanted immunological reaction in a human individual upon administration.

One embodiment of the present invention relates to a polypeptide comprising at least one nanobody wherein one or more amino acid residues have been substituted without substantially altering the antigen binding capacity.

The skilled person will recognize that the anti-RBD polypeptides of the present invention may be modified, and such modifications are within the scope of the invention. For example, the polypeptides may be used as drug carriers, in which case they may be fused to a therapeutically active agent, or their solubility properties may be altered by fusion to ionic/bipolar groups, or they may be used in imaging by fusion to an appropriate imaging marker, or they may comprise modified amino acids etc. They may also be prepared as salts. Such modifications which retain essentially the binding to RBD are within the scope of the invention.

As will be clear from the disclosure herein, it is also within the scope of the invention to use natural or synthetic analogs, mutants, variants, alleles, homologs and orthologs (herein collectively referred to as “analogs”) of the nanobodies of the invention as defined herein and in particular analogs of the nanobodies of SEQ ID NO's 22-28. Thus, according to one embodiment of the invention, the term “nanobody of the invention” in its broadest sense also covers such analogs.

Generally, in such analogs, one or more amino acid residues may have been replaced, deleted and/or added, compared to the nanobodies of the invention as defined herein. Such substitutions, insertions or deletions may be made in one or more of the framework regions and/or in one or more of the CDRs. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

Yet another modification may comprise the introduction of one or more detectable labels or other signal-generating groups or moieties, depending on the intended use of the labelled nanobody. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as 152 Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio isotopes (such as ³H, ¹²⁵I, ³²p, ³⁵S, ¹⁴C, ⁵¹Cr, ⁵⁷Co, ⁵⁸Co, ⁵⁹Fe, and ⁷⁵Se), metals, metals chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga or other metals or metallic cations that are particularly suited for use in in vivo, in vitro or in situ diagnosis and imaging, such as (¹⁵⁷Gd, ⁵⁵Mn, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, β-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled nanobodies and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethylenetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the nanobody of the invention to another protein, polypeptide or chemical compound that is bound to the other half of the binding pair, i.e. through formation of the binding pair. For example, a nanobody of the invention may be conjugated to biotin, and linked to another protein, polypeptide, compound or carrier conjugated to avidin or streptavidin. For example, such a conjugated nanobody may be used as a reporter, for example in a diagnostic system where a detectable signal-producing agent is conjugated to avidin or streptavidin. Such binding pairs may for example also be used to bind the nanobody of the invention to a carrier, including carriers suitable for pharmaceutical purposes. One non-limiting example is the case with liposomal formulations described by Cao and Suresh, Journal of Drug Targeting, 8, 4, 257 (2000). Such binding pairs may also be used to link a therapeutically active agent to the nanobody of the invention.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

As mentioned above, the invention also relates to proteins or polypeptides that essentially consist of at least one nanobody of the invention. By “essentially consist of is meant that the amino acid sequence of the polypeptide of the invention either is exactly the same as the amino acid sequence of a nanobody of the invention or corresponds to the amino acid sequence of a nanobody of the invention which has a limited number of amino acid residues, such as 1-20 ammo acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the nanobody.

Said amino acid residues may or may not change, alter or otherwise influence the (biological) properties of the nanobody and may or may not add further functionality to the nanobody.

According to another embodiment, a polypeptide of the invention comprises a nanobody of the invention, which is fused at its amino terminal end, at its carboxy terminal end, or both at its amino terminal end and at its carboxy terminal end to at least one further amino acid sequence, i.e. so as to provide a fusion protein comprising said nanobody of the invention and the one or more further amino acid sequences. Such a fusion will also be referred to herein as a “nanobody fusion”.

The one or more further amino acid sequence may be any suitable and/or desired amino acid sequences. The further amino acid sequences may or may not change, alter or otherwise influence the (biological) properties of the nanobody, and may or may not add further functionality to the nanobody or the polypeptide of the invention. Preferably, the further amino acid sequence is such that it confers one or more desired properties or functionalities to the nanobody or the polypeptide of the invention.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises a) at least one nucleic acid of the invention; operably connected to b) one or more regulatory elements, such as a promoter and optionally a suitable terminator; and optionally also c) one or more further elements of genetic constructs known per se; in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of the coding sequence (in which said coding sequence should be understood as being “under the control of said promotor). Generally, when two nucleotide sequences are operably linked, they will be in the same orientation and usually also in the same reading frame. They will usually also be essentially contiguous, although this may not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein and/or those used in the Examples.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localization of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression in bacterial cells, such as those mentioned herein and/or those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. Nos. 6,207,410, 5,693,492 and EP 1 085 089. Other examples will be clear to the skilled person. Reference is also made to the general background art cited above and the further references cited herein.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as those mentioned herein.

The nucleic acids of the invention and/or the genetic constructs of the invention may be used to transform a host cell or host organism, i.e. for expression and/or production of the nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism.

Generally, for the prevention and/or treatment of the diseases and disorders mentioned herein and depending on the specific disease or disorder to be treated, the potency of the specific nanobody and polypeptide of the invention to be used, the specific route of administration and the specific pharmaceutical formulation or composition used, the nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

It should also be noted that, when the nanobodies of the inventions contain one or more other CDR sequences than the preferred CDR sequences mentioned above, these CDR sequences can be obtained in any manner known per se, for example from nanobodies (preferred), V_{H} domains from conventional antibodies (and in particular from human antibodies), heavy chain antibodies, conventional 4-chain antibodies (such as conventional human 4-chain antibodies) or other immunoglobulin sequences directed against A-beta. Such immunoglobulin sequences directed against A-beta can be generated in any manner known per se, as will be clear to the skilled person, i.e. by immunization with A-beta or by screening a suitable library of immunoglobulin sequences with A-beta, or any suitable combination thereof. Optionally, this may be followed by techniques such as random or site-directed mutagenesis and/or other techniques for affinity maturation known per se. Suitable techniques for generating such immunoglobulin sequences will be clear to the skilled person, and for example include the screening techniques reviewed by Hoogenboom, Nature Biotechnology, 23, 9, 1105-1116 (2005). Other techniques for generating immunoglobulins against a specified target include for example the Nanoclone technology (as for example described in the non-prepublished U.S. provisional patent application 60/648,922), so-called SLAM technology (as for example described in the European patent application 0 542 810), the use of transgenic mice expressing human immunoglobulins or the well-known hybridoma techniques (see for example Larrick et al, Biotechnology, Vol. 7, 1989, p. 934). All these techniques can be used to generate immunoglobulins against A-beta, and the CDRs of such immunoglobulins can be used in the nanobodies of the invention, i.e. as outlined above. For example, the sequence of such a CDR can be determined, synthesized and/or isolated, and inserted into the sequence of a nanobody of the invention (e.g. so as to replace the corresponding native CDR), all using techniques known per se such as those described herein, or nanobodies of the invention containing such CDR's (or nucleic acids encoding the same) can be synthesized de novo, again using the techniques mentioned herein.

The invention will now be further described by means of the following non-limiting examples and figures.

### EXAMPLES

1) Material and Methods

Cell Lines

VERO-E6 (ATCC® CRL-1586), CaCO2 (ATCC® HTB-37) and 293T (ATCC® CRL-3216) cell are cultured in Dulbecco's Modified Eagle Medium (DMEM, Thermo Fisher Scientific, #12430112), supplied with 10% fetal bovine serum (Thermo Fisher Scientific, #26140079), 1% Penicillin-Streptomycin (Thermo Fisher Scientific, #15140148), and propagated at 37° C. in 5% CO 2. ExpiCHO Expression System was purchased from Thermo Fisher Scientific (#A29133).

Expression of Recombinant hACE2 and SARS-CoV-2 Spike RBD Protein

For hACE2-Fc, the extracellular domain of human hACE2 (1-740 aa) (GenBank: NM_021804.1) was amplified from a plasmid (HG10108-ACG, Sinobiologic), and fused to a human IgG1 Fc fragment with a (GSSSS) 3 linker (SEQ ID NO: 36). The whole CDS was cloned into a pCMV3 expression vector. The construct was expressed with ExpiCHO™ Expression System (A29133, Thermo Fisher Scientific) according to the manual. The protein was purified from culture supernatant by protein A affinity chromatography and stored at −70° C. in PBS buffer. For RBD constructs, amino acid 319-541 of SARS-CoV-2 S protein (GenBank: MN908947.3) was expressed with a signal peptide, MEFGLSWVFLVALFRGVQC (SEQ ID NO: 37), at the N-terminal, and a 6× his tag (for RBD-his) or a human IgG1 Fc fragment with (GSSSS) 3 linker (SEQ ID NO: 36) (for RBD-Fc) at the C-terminal. The whole CDS of these constructs were then cloned into a pCMV3 expression vector. For RBD-Fc, the construct was expressed in 293F cells transfected with PEI MAX™ (24765-1, Polysciences, Inc.) according to the manual. The protein was purified from culture supernatant by protein A affinity chromatography and stored at −70° C. in PBS buffer. For RBD-His, the construct was expressed with ExpiCHO™ Expression System. The protein was purified from culture supernatant by Nickel affinity chromatography and stored at −70° C. in PBS buffer as well.

For Nickel affinity chromatography, the culture supernatant from transient expression product was clarified by centrifuge at 3000 g for 10 min and was mixed with equal volume of 20 mM imidazole, 500 mM NaCl, 20 mM Tris pH8.0. The protein was purified with HisTrap™ HP column (17524701, Cytiva Inc., Marlborough MA, USA), and was eluted with 500 mM imidazole, 500 mM NaCl, 20 mM Tris pH8.0. The eluted fraction was concentrated and desalted into PBS with Amicon® Ultra-15 centrifugal unit (MilliporeSigma Life Science Center, Burlington, Massachusetts, USA) with appropriate MWCO.

For polishing with hydroxyapatite chromatography, sample was buffer changed into 5 mM sodium phosphate, 20 mM MES, pH6.6, and was loaded on self-packed columns with Ca++Pure HA resin (45039, Tosoh Bioscience LLC, PA, Japan); the protein was eluted by a gradient elution with 400 mM sodium phosphate, 20 mM MES, pH6.6, and the targeted fraction was concentrated and desalted into PBS with Amicon® Ultra-15 centrifugal unit with appropriate MWCO.

All proteins were checked by SDS-PAGE and HPLC with TSKgel G3000SWXL (08541, Tosoh Bioscience LLC, PA, Japan) for purity.

Construction of Phage Displayed Synthetic V_{HH} Library by Oligonucleotide-Directed Mutagenesis

Three CDRs of the V_{HH} template (a humanized V_{HH} from the V germline gene, IGHV3S1*01, of Camelus dromedaries) were mutagenized using synthesized oligonucleotides encoding tailored diversity of amino acids. In brief, mutagenic oligonucleotides for each CDR were mixed and phosphorylated by T4 polynucleotide kinase (New England BioLabs) in the buffer comprising 70 mM Tris-HCl (pH 7.6), 10 mM MgCl₂, 1 mM ATP and 5 mM dithiothreitol (DTT) at 37° C. for 1 h. The single stranded DNA template containing uracil was obtained by using the defective E. coli strain, CJ236. The phosphorylated oligonucleotides were then annealed to the uracilated single-stranded DNA template of V_{HH}, at a molar ratio of 3:1 (oligonucleotide:ssDNA), by heating the mixture at 90° C. for 2 min, followed by a temperature decrease of 1° C./min to 20° C. in a thermal cycler. Subsequently, the template with annealed oligonucleotides was incubated in the buffer containing 0.32 mM ATP, 0.8 mM dNTPs, 5 mM DTT, T4 DNA ligase and T7 DNA polymerase (New England BioLabs) for the in vitro synthesis of new DNA strain bearing CAR mutations. After overnight incubation at 20° C., the synthesized dsDNA was desalted and concentrated, then electroporated into E. coli strain, ER2738, followed by the M13KO7 helper phage infection and overnight culturing. Finally, the phage displaying nanobodies as a library in the culture medium was harvested and precipitated by polyethylene glycol (PEG)/NaCl for further use.

Screening of Anti-RBD Antibodies

RBD specific VHHs were identified from the screening (bio panning) of phage displayed synthetic V_{HH} library. Recombinant RBD-Fc (2˜5 μg per well) was coated in PBS buffer (pH 7.4) in NUNC 96-well Maxisorb immunoplates (NUNC) overnight at 4° C. and then blocked with 5% skim milk in PBST [0.05% (v/v) Tween 20] for 1 h. After blocking, 100 μL of resuspended PEG/NaCl-precipitated phage library (10¹¹⁻¹² cfu/mL in blocking buffer) was incubated in each well for 1 h under gentle shaking. The plate was washed 10 times with 250 μL PBST and 2 times with 200 μL PBS. The bound phages were eluted with 100 μL of 0.1 M HCl/glycine (pH 2.2) per well, immediately neutralized with 8 μL of 2 M Tris-base buffers (pH 9.1). The eluted phages were mixed with 1 mL of E. coli ER2738 (A600 nm=0.6) for 30 min at 37° C.; uninfected bacteria were eliminated by adding ampicillin. After 30 minutes, the bacterial culture was infected with 100 μL M13K07 helper phage (˜10¹¹ CFU in total) at 37° C. for 1 h. The infected ER2738 cells were finally mixed with 2×YT medium containing 50 μg/mL kanamycin and 100 μg/mL ampicillin and cultured for overnight at 37° C. with vigorous shaking. Next day, the amplified phage pool was precipitated with 20% PEG/NaCl and resuspended in PBS for the next round of panning.

After 2-3 rounds of selection-amplification cycle, single colonies were randomly picked into deep 96 well culture plate; each well contained 850 μL 2YT with 100 μg/mL ampicillin. After 3 h incubation at 37° C. with shaking, 50 μL M13KO7 (˜5×10¹⁰ CFU in total) was added to each well of the plate. One hour later, 100 μL 2YT containing 500 μg/mL kanamycin was added and cultured for overnight at 37° C. with vigorous shaking. Next day, the cultures were centrifuged at 3000 g for 10 min at 4° C. 50 μL culture medium and 50 μL 5% skim milk/PBST was added to a corresponding well of 96-well Maxisorb immunoplates (NUNC) pre-coated with RBD-Fc or Fc protein (1 μg/ml) and blocked with 5% skim milk/PB ST. After 1 h incubation at room temperature, the plates were washed with PBST and the phage binding to antigens was detected by M13-HRP antibody (1:3000, Sinobiologic) with 1 h incubation. After another PBST wash, the positive signals were developed by 3, 3′, 5, 5′-tetramethyl-benzidine peroxidase substrate (Kirkegaard & Perry Laboratories), quenched with 1.0 M HCl and read spectrophotometrically at 450 nm. Positive clones were selected by the following criteria: ELISA OD450>0.2 for the RBD-Fc coated well; OD450<0.1 for Fc well. Unique clones were determined by sequencing of the V_{HH} gene harbored in the phagemids.

Screening of Nanobodies Blocking the Interaction of RBD and hACE2

To produce PCR product for cell transient expression, two-step PCR were performed. Unique VHH sequences from phage ELISA and sequencing results were PCR amplified from phage supernatant. Then one fragment containing a CMV promoter and a human trypsinogen-2 signal peptide and the other containing a 12aa linker (GSGGGGSGGGGS) (SEQ ID NO: 38), a human IgG1-Fc and a SV40 polyA signal were amplified, then fused to the 5 and 3 prime ends of the VHH gene by overlapping PCR, respectively. The PCR products of the expression cassette for Fc tagged nanobodies were expressed by ExpiCHO Expression System. Five days later, the culture medium containing Fc tagged nanobodies were collected and subjected to RBD blocking ELISA screening.

For blocking ELISA, a 96-well Maxisorp plate was coated with hACE2-Fc (2 μg/ml, 100 μL per well) at 4° C., overnight and then blocked with blocking buffer (2% BSA in PBS) for 2 h. 50 uL of VHH-Fc cell supernatant (expression product of PCR fragment) was added to 50 uL of PBT, which containing RBD-his (40 ng/ml). VHH72-Fc and a non-related V_{HH}-Fc (produced with the same method) were used as a positive and negative control, respectively. hACE2-Fc (2 μg/ml in PBS) was also as a reference. After 1 h incubation with gentle shaking, 90 PLL of the mixtures were transferred to the BSA-blocked plate and incubated for 20 min. RBD-His binding to the plate was detected with anti-His tag mouse monoclonal antibody (1:3000 dilution, SinoBiological, 105327-MM02T) and followed by an HRP conjugated anti-mouse IgG (H+L) Goat antibody (Beyotime, A0216). The RBD binding signals were developed by 3, 3′, 5, 5′-tetramethyl-benzidine peroxidase substrate (Kirkegaard & Perry Laboratories), quenched with 1.0 M HCl and read at OD 450 nm by a spectrophotometer.

Expression and Purification of Fc Tagged Nanobodies

For Fc tagged VHH constructs, the specific VHH domain was amplified from original phage clone by PCR. The VHH gene fragments were sub-cloned into the pCMV3 expression vector with an upstream signal peptide of human trypsinogen-2 and a downstream human IgG1Fc fragment with a linker (GSGGGGSGGGGS) (SEQ ID NO: 38). The constructs were expressed in ExpiCHO™ Expression System according to the manual. The VHH-Fc proteins expressed in the culture medium were clarified by centrifuge at 3000 g for 10 min and mixed with equal volume of 1.5 M Glycine, 3 M NaCl, pH 8.9. The protein was purified with HiTrap™ MabSelect™ SuRe™ column (11003494, Cytiva Inc.), and eluted with 20 mM acetic acid, pH 3.5. The acid eluted fraction was neutralized with 1M Tris-HCl, pH 9.0 and concentrated/desalted into PBS with Amicon® Ultra-15, PLTK Ultracel-PL membrane (MilliporeSigma Life Science Center, Burlington, Massachusetts, USA) with appropriate MWCO.

RBD Binding Assay

ELISA was used to test the RBD specificity of selected nanobodies in serial dilution manner. In brief, the RBD-Fc antigen (0.2 μg per well) were coated in PBS buffer (pH7.4) on NUNC 96-well Maxisorb immunoplates overnight at 4° C. and blocked with 5% skim milk in PBST for 1 h. 100 μL VHHs prepared at serial concentrations in PBST with 2.5% milk were added to each well and incubated for 1 h under gentle shaking. The plate was washed with PBST and then added with 100 μL 1: 2000-diluted anti-human IgG conjugated with horse-radish peroxidase for another 1 h. The plates were washed with PBST buffer and twice with PBS, developed for 3 min with 3, 3′, 5, 5′-tetramethyl-benzidine peroxidase substrate (Kirkegaard & Perry Laboratories), quenched with 1.0 M HCl and read spectrophotometrically at 450 nm.

Surface Plasmon Resonance (SPR) Assay

The affinity of anti-RBD nanobodies and RBD antigen was measured with SPR. A biosensor chip, Series S Sensor Chip Protein A (Cat. #29127556, GE), was used to affinity-capture a certain amount of Fc tagged nanobodies to be tested and then flow through a series of COVID-19 S.P. RBD (Cat. #40592-V08B, SB) under a concentration gradient on the surface of the chip {dilution ratio: 2; concentration levels: at least 5 (excluding curves with irregularities or high background)}. A Biacore 8K (Serial NO. 29327020-2473040, GE) instrument was used to detect the reaction signal in real time to obtain the association and dissociation curves.

The Biacore 8K was also used to determine the blocking effect of anti-RBD nanobodies on the binding of hACE2 to RBD antigen. The biosensor chip Series S Sensor Chip Protein A (Cat. #29127556, GE) was used to affinity-capture a certain amount of Fc tagged nanobodies to be tested. Next, 50 nM of RBD (Cat. #40592-V08B, SB) was injected followed by a flow through of 100 nM human hACE2 (Cat. #1010B-H08H, SB) onto the surface of the chip. The Biacore 8K was used to detect the reaction signal in real time to obtain the binding and dissociation curves (theoretical hACE2 Rmax>220 RU and kinetically simulated hACE2 binding >160 RU for all). The buffer used in the experiment was HBS-EP+ solution (pH 7.4, Cat. #BR100669, GE). The data obtained in the experiment was fitted with Biacore Insight Evaluation Software v3.0, GE software with a (1:1) binding model to obtain the affinity value.

Pseudovirus Neutralization

Pseudovirus neutralization assay was measured by reduction of Luciferase activity as described [39]. Briefly, the pseudovirus bearing SARS-CoV-2 S protein was produced by co-transfection of 293T with plasmids expressing SARS-CoV-2 S protein and pNL-4-3-Luc.-R-E. The pseudovirus was harvested, filtered and stored in −80° C. Before infection of CaCO2 cells, the pseudovirus was incubated with serial diluted nanobodies for 30 min at room temperature. Luciferase activities were measured after 48 hours post infection according to manual of Bright-Glo™ Luciferase Assay System. The non-infected cells were considered as 100% inhibition, and cells only infected with the virus were set as 0% inhibition. The EC 50 values of the nanobodies were calculated with non-linear regression using GraphPad Prism 5 (GraphPad Software, Inc., San Diego, CA, USA).

SARS-CoV-2 Neutralization Assay

The SARS-CoV-2 (strain IVCAS 6.7512) was provided by the National Virus Resource, Wuhan Institute of Virology, Chinese Academy of Sciences. All SARS-CoV-2 live virus related experiments were approved by the Biosafety Committee Level 3 (ABSL-3) of Wuhan University. All experiments involving SARS-CoV-2 were performed in the BSL-3 and ABSL-3 facilities.

Briefly, nanobodies were serially diluted in culture medium and 100 μl was mixed with 100 μl (1000 PFU) SARS-CoV-2 for 30 min. The mixture was then added to Vero E6 cells (ATCC number: CRL-1586) in 48-well plates and incubated for 24 hours. Then, TRIzol (Invitrogen) was added to inactivate SARS-CoV-2 viruses and RNA was extracted according to the manufacturer's instructions. First-strand cDNA was synthesized using the PrimeScript RT kit (TakaRa). A real time quantitative PCR was used to detect the presence of SARS-CoV-2 viruses by the primers (Table 1).

TABLE 1
Primers for RT-PCR of SARS-CoV-2

| | |
|---|---|
| SARS2-N-F | TAATCAGACAAGGAACTGATTA |
| | (SEQ ID NO: 39) |
| SARS2-N-R | CGAAGGTGTGACTTCCATG (SEQ ID NO: 40) |
| SARS2-N-P | GCAAATTGTGCAATTTGCGG (SEQ ID NO: 41) |
| hGAPDH-F | CAGCCTCAAGATCATCAGCA (SEQ ID NO: 42) |
| hGAPDH-R | TGTGGTCATGAGTCCTTCCA (SEQ ID NO: 43) |
| hGAPDH-P | CTGCTTAGCACCCCTGGCCA (SEQ ID NO: 44) |
| hACE2-F | CATTGGAGCAAGTGTTGGATCTT |
| | (SEQ ID NO: 45) |
| hACE2-R | GAGCTAATGCATGCCATTCTCA |
| | (SEQ ID NO: 46) |
| hACE2-P | CTTGCAGCTACACCAGTTCCCAGGCA |
| | (SEQ ID NO: 47) |
| mGAPDH-F | TGCACCACCAACTGCTTAG (SEQ ID NO: 48) |
| mGAPDH-R | GGATGCAGGGATGATGTTC (SEQ ID NO: 49) |
| mGAPDH-P | CAGAAGACTGTGGATGGCCCCTC |
| | (SEQ ID NO: 50) |

The relative number of SARS-CoV-2 viral genome copies was determined using a TaqMan RT-PCR Kit (Yeason). To accurately quantify the absolute number of SARS-CoV-2 genome copies, a standard curve was prepared by measuring the SARS-CoV-2 N gene constructed in the pCMV-N plasmid. All SARS-CoV-2 genome copy numbers were normalized to GAPDH expression in the same cell.

Assay for Protection of K18-hACE2 Transgenic Mice Against SARS-CoV-2 Mediated by Nanobodies

K18-hACE2 transgenic mice expressing human ACE2 driven by the human epithelial cell cytokeratin-18 (K18) promoter were purchased from Gempharmatech and housed in ABSL-3 pathogen-free facilities under 12-h light-dark cycles with ad libitum access to food and water. All animal experiments were approved by the Animal Care and Use Committee of Wuhan University. Age-matched (9-10 week-old) female mice were grouped for infection of nanobodies (0.5 mg/kg). One day later, mice were inoculated with 6×10⁴ PFU of SARS-CoV-2 by the intranasal route. Body weights were monitored at 3 and 6 d.p.i (day post infection). Animals were sacrificed at 3 or 6 d.p.i according to the protocol, and tissues were harvested for pathologic and histologic analysis.

Plaque Assay of Lung Tissue Homogenates

The right lung was homogenized in 1 mL PBS using a Tissue Cell-destroyer 1000 (NZK LTD). Vero E6 (ATCC number: CRL-1586) cells were cultured to determinate viral titer. Briefly, serial 10-fold dilutions of samples were added into monolayer cells. After adsorption at 37° C., the virus inoculum was removed and cells were washed with PBS twice, then DMEM containing 5% FBS and 1.0% methylcellulose was supplemented. Plates were incubated for 2 days until obvious plaques can be observed. Cells were stained with 1% crystal violet for 4 h at room temperature. Plaques were counted and viral titers were defined as PFU/ml.

Histological Analysis

Lung samples were fixed with 4% paraformaldehyde, paraffin embedded and cut into 3.5-mm sections. Fixed tissue samples were used for hematoxylin-eosin (H&E) staining and indirect immunofluorescence assays (IFA). Histological analysis was performed by Wuhan Servicebio Technology Co., Ltd. For IFA, Anti-hACE2 antibody and anti-SARS-CoV/SARS-CoV-2 Nucleocapsid Antibody (Cat: 10108-RP01 and 40143-MM05, SinoBiological) were added as primary antibodies. The image information was collected using a Pannoramic MIDI system (3DHISTECH, Budapest) and FV1200 confocal microscopy (Olympus).

Data Analysis

All the assays were conducted with at least duplicated biologic repeats. Results were presented by representative data or mean±SEM with indicated numbers of replication. All data was analyzed by XLfit (IDBS, Boston, MA 02210) or Prism 5(GraphPad Software, San Diego, CA 92108).

2) Results

Generation of Neutralizing Nanobodies Against SARS-COV2

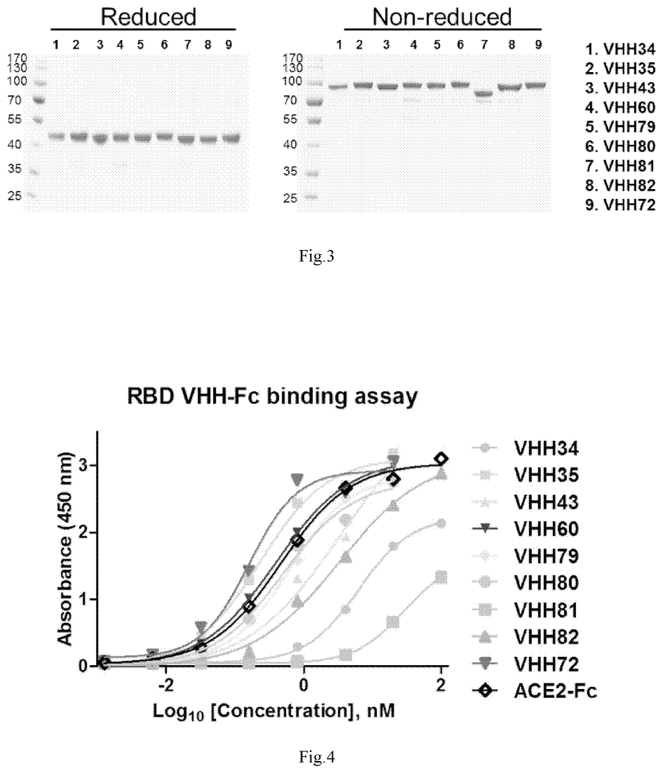

To generate the nanobodies that neutralize SARS-CoV-2, pre-designed synthetic nanobody library technology was used. The complementary determination region (CDR) sequences with tailored diversity were genetically engineered into an optimized humanized nanobody framework by a high-speed DNA mutagenesis method. The resulting nanobodies were displayed on phage as a library to be screened for the binders against the recombinant RBD protein (FIG. 1). Phage displayed synthetic nanobody library with the size around 10¹⁰ was first screened against RBD. After 3 rounds of phage selection, the individual clone enriched from phage pool was amplified by PCR and cloned into CMV promoter driven mammalian expression ExpiCHO system for 2^{nd} round ELISA-based selection by competing with hACE2 for RBD binding (FIG. 1). 78 nanobodies were finally identified with unique sequences including 7 nanobodies displayed blocking ability similar to VHH72 (FIG. 2). All these 7 antibodies were then expressed with Fc-tag for further 10 characterization (Table 2). After protein A column purification, nanobodies were present as monomers of ˜40 kDa in reducing gel as shown in FIG. 3.

TABLE 2
CDR sequences of 7 nanobodies

| Clones | CDR1 | CDR2 | CDR3 |
|---|---|---|---|
| VHH34 | GFIYSFNIMG | SINWFSDITYY | AYLLRGDDRYYATYSYWG (SEQ ID NO: 15) |
| | (SEQ ID NO: 13) | (SEQ ID NO: 14) | |
| VHH35 | GSIYSFNFMG | TINSFDDITYY | VLGERTGISYGSAFDYWG (SEQ ID NO: 6) |
| | (SEQ ID NO: 4) | (SEQ ID NO: 5) | |
| VHH43 | GFISDADIMG | SINSYDSITYY | VRVHSRDFSYWG (SEQ ID NO: 18) |
| | (SEQ ID NO: 16) | (SEQ ID NO: 17) | |
| VHH60 | GRTFRVNLMG | SINGFDDITYY | AYDSDYDGRLFNYWG (SEQ ID NO: 3) |
| | (SEQ ID NO: 1) | (SEQ ID NO: 2) | |
| VHH79 | GFTSRNYFMG | TINSLSSITYY | VYTPTTGPGEGSYTPWHDYWG (SEQ ID |
| | (SEQ ID NO: 7) | (SEQ ID NO: 8) | NO: 9) |
| VHH80 | GFISNFNLMG | TINSFDDITYY | AEVRSSLDYALWTSRRSAFSYWG (SEQ ID |
| | (SEQ ID NO: 10) | (SEQ ID NO: 11) | NO: 12) |
| VHH82 | GFIYSFNIMG | SISSYDDITYY | AYLLRGDDRYYATYSYWG (SEQ ID NO: 21) |
| | (SEQ ID NO: 19) | (SEQ ID NO: 20) | |

Synthetic Nanobodies Specifically Bind to SARS-CoV-2 RBD with High Affinity

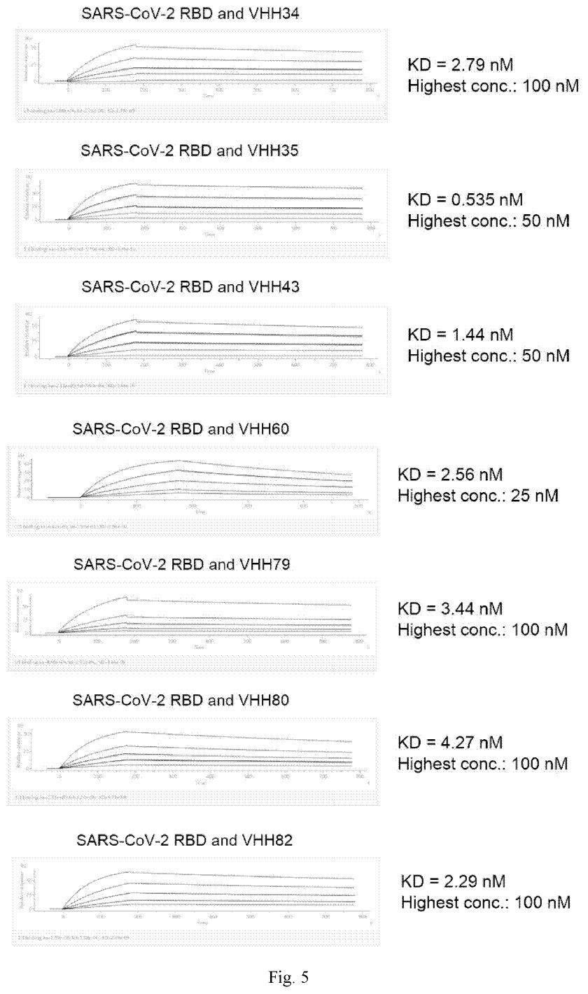

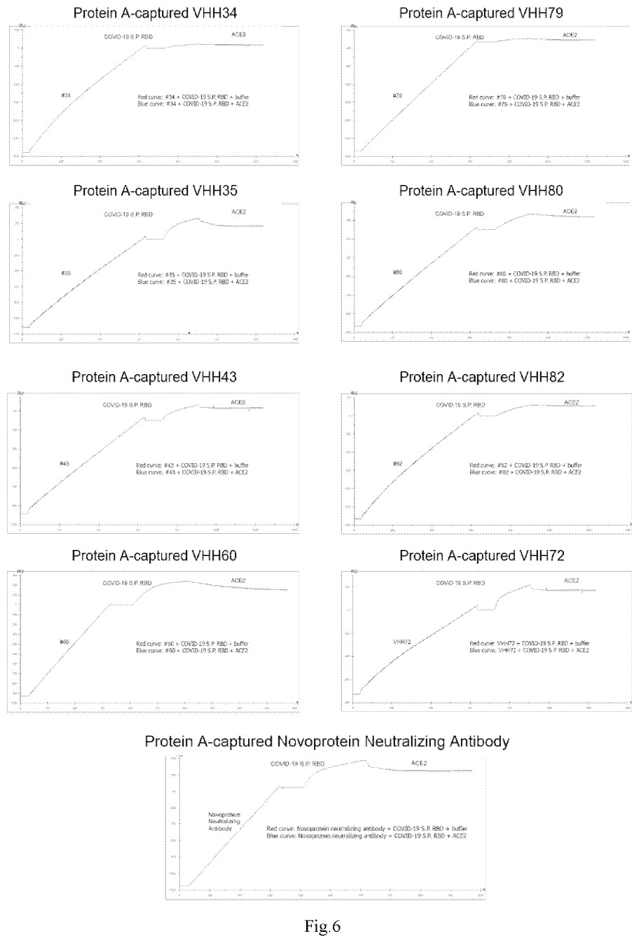

ELISA was conducted to evaluate the binding capacity of the nanobodies to its original target RBD. VHH35, VHH60, VHH79 and VHH80 showed slightly higher or similar affinity to the recombinant RBD protein compared to the VHH72 reference (FIG. 4). Next, the affinity was determined by Surface Plasmon Resonance (SPR). Among the tested nanobodies, VHH35 exhibited the lowest K D of 0.535 nM to RBD, the rest of nanobodies all bound to RBD with single-digit nanomolar Dissociation constants (FIG. 5). To further confirm the blocking effect of the nanobodies to the RBD/hACE2 interaction, SPR was also used to measure the affinity of hACE2 to RBD after the RBD was first bound by nanobodies captured on the protein A chip. No binding curve of hACE2 to RBD could be detected when the RBD were pre-occupied by the nanobodies (FIG. 6). The ELISA and SPR data indicated that these 7 nanobodies were able to block the binding of hACE2 to RBD.

VHH60 Suppresses Infection and Amplification of SARS-CoV-2 Virus In Vitro and In Vivo

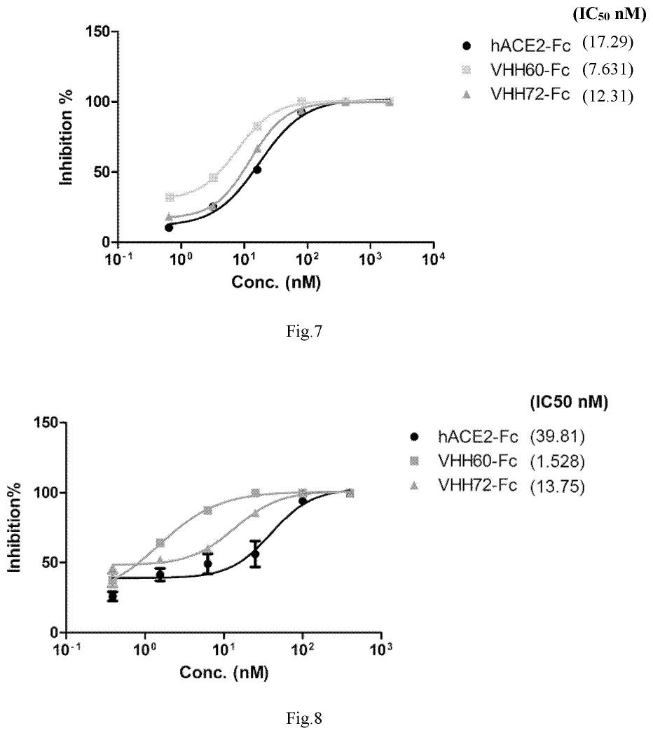

To investigate the neutralizing activity of nanobodies, a pseudovirus based cell entry assay was deployed. Pseudoviruses bearding S protein and luciferase were incubated with various concentrations of 4 nanobodies having strong binding affinity for 30 min prior to infect Caco-2 cells. The results of Luciferase activity measured 48 h post infection suggested that the nanobodies provided robust protection compared to the VHH72 and hACE2 controls. In particular, the VHH60 provided the best protection with an IC50 of 7.631 nM (FIG. 7).

To further evaluate the antiviral effect of VHH60, authentical SARS-CoV-2 virus was used on Vero-E6 cell in vitro. Virus was premixed with serially diluted nanobodies for 30 min, then added to the Vero-E6 cell to propagate for 24 hours. Viral RNA level was measured by RT-PCR. The data showed that VHH60 inhibited viral infection at an IC50 of 1.528 nM, which was 8-fold lower than the IC50 of the reference VHH72 (13.75 nM) (FIG. 8).

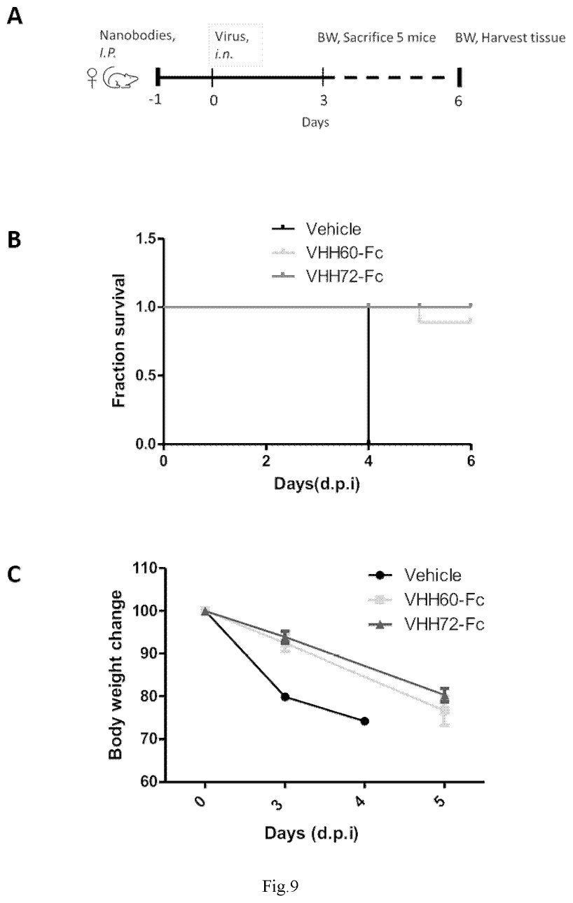

Then the antiviral potential of VHH60 was investigated in vivo. 10 female K18-hACE2 transgenic mice per group expressing human ACE2 were intraperitoneally administrated with nanobodies or controls (Vehicle: PBS) at 0.5 mg/kg, at 24 hours prior to inoculation with authentical SARS-CoV-2 virus intranasally. 5 mice of each group were sacrificed for pathologic analysis at 3 d.p.i. as planned (FIG. 9A). The remaining mice of the vehicle group all died at 4 d.p.i. (5 out of 5, observed at day 5), but mice treated with the nanobodies (VHH60 and VHH72) survived up to 6 days excepted one mouse in the VHH60 group that died at 5 d.p.i. (1 out of 5, observed at day 6) which could be considered as a normal variation (FIG. 9B). All VHH60 and VHH72 treated mice were sacrificed at day 6 post infection because the body weight of the mice dropped up to 25%, which met the termination criteria according to the IACUC protocol. Consistent with previous report that virus infection could cause body weight loss [40], also it was observed that at 3 d.p.i. the body weight of mice from the vehicle group had dropped 20%. In contrast the body weight of mice treated with VHH60 and VHH70 decreased only slightly (FIG. 9C).

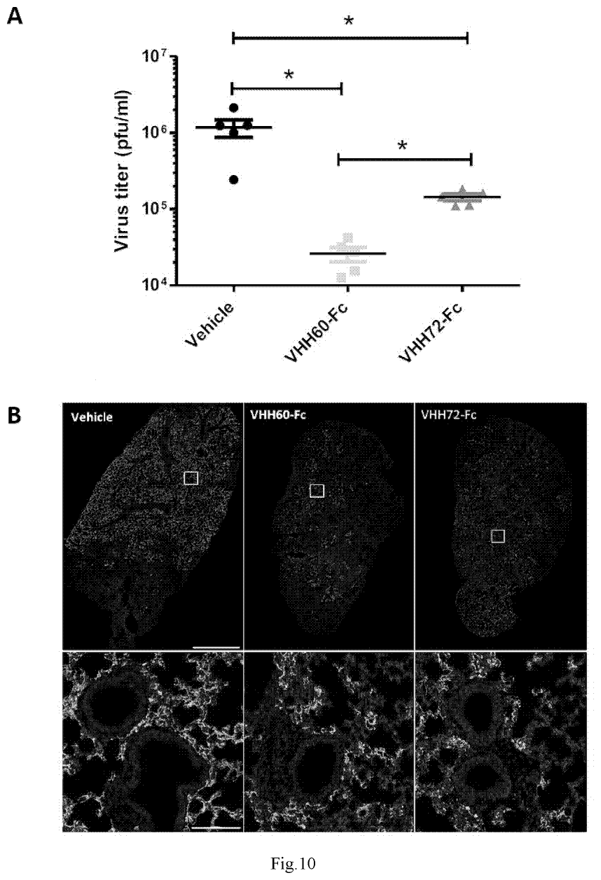

To more accurately assess the protective effect, viral load was evaluated at 3 d.p.i when all mice including the vehicle group were still alive. Virus titer from lung in VHH60 treated group was significantly suppressed to a level which was 45-fold lower than that of the vehicle and 9-fold lower than that of the VHH72 group, respectively (FIG. 10A). Immunofluorescent data clearly confirmed that the viral particles represented by green nucleocapsid staining were much fewer in VHH60 and VHH72 groups compared to those in the vehicle group (FIG. 10B). There is no significant difference of red signal from ACE2 staining was observed, which could exclude the possibility that virus titer was affected by the ACE2 level.

Together, the results strongly support that VHH60 is highly efficacious to restrain infection and proliferation of SARS-CoV-2 both in vitro and in vivo, ameliorate disease progress and improve health.

VHH60 Blocks the Infection of Mutated Pseudovirus

Given the high mutagenic capacity of SARS-CoV-2 as an RNA virus, escape mutations and variants resistant to current antibodies or vaccines have been studied and described [41-48]. Antibody cocktails or broadly neutralizing antibodies have stood out and gained more attentions as a new modality to combat COVID19 [49, 50].

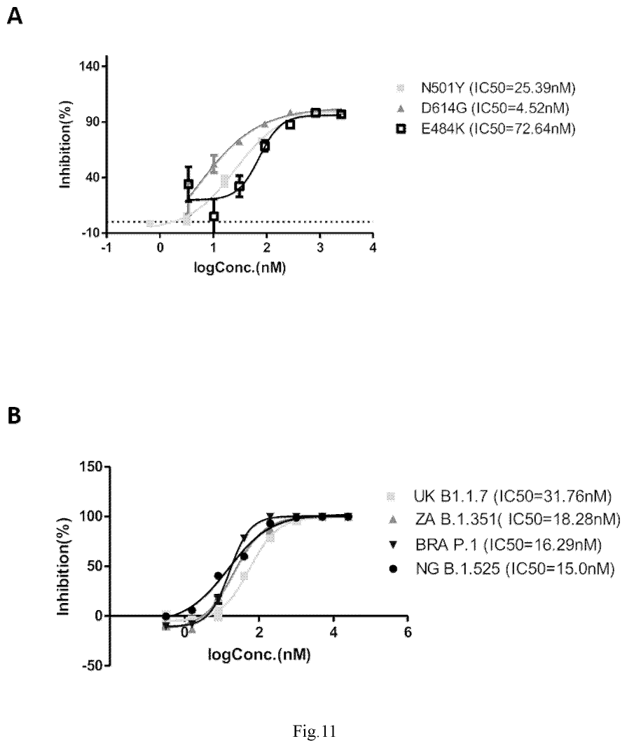

In addition to the wildtype S protein, mutants and variants carrying more than one mutation were tested with VHH60 in pseudoviral entry assays. VHH60 inhibited all single mutants E484K, N501Y and D614G at nanomolar IC50 level (FIG. 11A). Strikingly, VHH60 also exhibited robust activity to suppress variants B1.1.7 (IC50=31.76 nM), B.1.351 (IC50=18.28 nM), P.1 (IC50=16.29 nM), and B.1.525 (IC50=15.0 nM) at IC50 values close to or even better than that for wildtype S protein (FIG. 11B).

### CONCLUSION

The results evidently demonstrate that nanobodies of the invention directly bind to SARS-CoV-2 RBD and effectively block virus infection in cells. VHH60 potently competes with hACE2 to bind RBD and effectively blocks SARS-CoV-2 virus interaction with its host receptor to prevent infection in cell lines and a mouse model. Importantly VHH60 also maintains a broad capacity to neutralize multiple escape mutants and variants. These nanobodies have been viewed as a revolutionary discovery for antibody-based therapies. Unlike traditional monoclonal antibodies, nanobody has a natural advantage for prophylactic usage, which is especially important as SARS-CoV-2 spreads via droplets and aerosol [38]. The highly encouraging outcome of the study lands strong support for the further development of the nanobodies for ultimate therapeutic applications.

Furthermore, these nanobodies may be used for diagnostic purposes, or as an adaptor to make heterodimer or polymers with other nanobodies or reagents having more promising antiviral potentials. It should be apparent to those skilled in the art that variations and modifications of the present invention may be made within the scope or spirit of the present invention. Therefore, it is to be understood that the invention is not limited to the specific embodiments disclosed and that modifications and other embodiments are intended to be included within the scope of the appended claims. Although specific terms are employed herein, they are used in a generic and descriptive sense only and not for purposes of limitation.

### REFERENCES

1. Jing Qin, C. Y., Qiushi Lin, Taojun, Shicheng Yu, Xiao-Hua Zhou, Estimation of incubation period distribution of COVID-19 using disease onset forward time: a novel cross-sectional and forward follow-up study. Science Advances, 2020. 6(33).
2. Lauer, S. A., et al., The Incubation Period of Coronavirus Disease 2019 (COVID-19) From Publicly Reported Confirmed Cases: Estimation and Application. Ann Intern Med, 2020. 172(9): p. 577-582.
3. He, X., et al., Temporal dynamics in viral shedding and transmissibility of COVID-19. Nat Med, 2020. 26(5): p. 672-675.
4. He, J., et al., Proportion of asymptomatic coronavirus disease 2019: A systematic review and meta-analysis. J Med Virol, 2020.
5. Beigel, J. H., et al., Remdesivir for the Treatment of Covid-19 —Final Report. N Engl J Med, 2020.
6. FDA Approves First Treatment for COVID-19. https://www.fda.govinews-events/press-announcements/fda-approves-first-treatment-covid-19, 2020.
7. Chen, Y., Q. Liu, and D. Guo, Emerging coronaviruses: Genome structure, replication, and pathogenesis. J Med Virol, 2020.
8. Wu, F., et al., A new coronavirus associated with human respiratory disease in China. Nature, 2020. 579(7798): p. 265-269.
9. Lu, R., et al., Genomic characterisation and epidemiology of 2019 novel coronavirus: implications for virus origins and receptor binding. Lancet, 2020. 395(10224): p. 565-574.
10. Zhou, P., et al., A pneumonia outbreak associated with a new coronavirus of probable bat origin. Nature, 2020. 579(7798): p. 270-273.
11. Hoffmann, M., et al., SARS-CoV-2 Cell Entry Depends on ACE2 and TMPRSS2 and Is Blocked by a Clinically Proven Protease Inhibitor. Cell, 2020. 181(2): p. 271-280 e8.
12. Shang, J., et al., Cell entry mechanisms of SARS-CoV-2. Proc Natl Acad Sci USA, 2020. 117(21): p. 11727-11734.
13. Cantuti-Castelvetri, L., et al., Neuropilin-1 facilitates SARS-CoV-2 cell entry and infectivity. Science (New York, N Y), 2020.
14. Daly, J. L., et al., Neuropilin-1 is a host factor for SARS-CoV-2 infection. Science (New York, N Y), 2020.
15. Clausen, T. M., et al., SARS-CoV-2 Infection Depends on Cellular Heparan Sulfate and ACE2. Cell, 2020.
16. Shang, J., et al., Structural basis of receptor recognition by SARS-CoV-2. Nature, 2020. 581(7807): p. 221-224.
17. Wrapp, D., et al., Cryo-EM structure of the 2019-nCoV spike in the prefusion conformation. Science, 2020. 367(6483): p. 1260-1263.
18. Cai, Y., et al., Distinct conformational states of SARS-CoV-2 spike protein. Science, 2020. 369(6511): p. 1586-1592.
19. Yan, R., et al., Structural basis for the recognition of SARS-CoV-2 by full-length human ACE2. Science, 2020. 367(6485): p. 1444-1448.
20. Lamers, M. M., et al., SARS-CoV-2 productively infects human gut enterocytes. Science, 2020. 369(6499): p. 50-54.
21. Yang, L., et al., A Human Pluripotent Stem Cell-based Platform to Study SARS-CoV-2 Tropism and Model Virus Infection in Human Cells and Organoids. Cell Stem Cell, 2020. 27(1): p. 125-136 e7.
22. Thaweerat, W., Current evidence on pancreatic involvement in SARS-CoV-2 infection. Pancreatology, 2020. 20(5): p. 1013-1014.
23. Chu, H., et al., Comparative tropism, replication kinetics, and cell damage profiling of SARS-CoV-2 and SARS-CoV with implications for clinical manifestations, transmissibility, and laboratory studies of COVID-19: an observational study. Lancet Microbe, 2020. 1(1): p. e14-e23.
24. Sungnak, W., et al., SARS-CoV-2 entry factors are highly expressed in nasal epithelial cells together with innate immune genes. Nat Med, 2020. 26(5): p. 681-687.
25. Renn, A., et al., Fruitful Neutralizing Antibody Pipeline Brings Hope To Defeat SARS-Cov-2. Trends Pharmacol Sci, 2020. 41(11): p. 815-829.
26. Premkumar, L., et al., The receptor binding domain of the viral spike protein is an immunodominant and highly specific target of antibodies in SARS-CoV-2 patients. Sci Immunol, 2020. 5(48).
27. Noy-Porat, T., et al., A panel of human neutralizing mAbs targeting SARS-CoV-2 spike at multiple epitopes. Nat Commun, 2020. 11(1): p. 4303.
28. Zost, S. J., et al., Potently neutralizing and protective human antibodies against SARS-CoV-2. Nature, 2020. 584(7821): p. 443-449.
29. Hussain, A., et al., Targeting SARS-CoV2 Spike Protein Receptor Binding Domain by Therapeutic Antibodies. Biomed Pharmacother, 2020. 130: p. 110559.
30. Chan, K. K., et al., Engineering human ACE2 to optimize binding to the spike protein of SARS coronavirus 2. Science, 2020. 369(6508): p. 1261-1265.
31. Cao, L., et al., De novo design of picomolar SARS-CoV-2 miniprotein inhibitors. Science, 2020. 370(6515): p. 426-431.
32. Martinez-Delgado, G, Inhaled nanobodies against COVID-19. Nat Rev Immunol, 2020. 20(10): p. 593.
33. Schoof, M., et al., An ultra-high affinity synthetic nanobody blocks SARS-CoV-2 infection by locking Spike into an inactive conformation. bioRxiv, 2020.
34. Xiang, Y., et al., Versatile, Multivalent Nanobody Cocktails Efficiently Neutralize SARS-CoV-2. bioRxiv, 2020.
35. Muyldermans, S., Nanobodies: natural single-domain antibodies. Annu Rev Biochem, 2013. 82: p. 775-97.
36. Morrison, C., Nanobody approval gives domain antibodies a boost. Nat Rev Drug Discov, 2019. 18(7): p. 485-487.
37. Wrapp, D., et al., Structural Basis for Potent Neutralization of Betacoronaviruses by Single-Domain Camelid Antibodies. Cell, 2020. 181(5): p. 1004-1015 e15.
38. Transmission of SARS-CoV-2: implications for infection prevention precautions. https://www.who.int/news-room/commentaries/detail/transmission-of-sars-cov-2-implications-for-infection-prevention-precautions, 2020.
39. Nie, J., et al., Establishment and validation of a pseudovirus neutralization assay for SARS-CoV-2. Emerg Microbes Infect, 2020. 9(1): p. 680-686.
40. Rogers, T. F., et al., Isolation of potent SARS-CoV-2 neutralizing antibodies and protection from disease in a small animal model. Science, 2020. 369(6506): p. 956-963.
41. Jangra, S., et al., SARS-CoV-2 spike E484K mutation reduces antibody neutralisation. Lancet Microbe, 2021.
42. Liu, H., et al., The basis of a more contagious 501Y.V1 variant of SARS-COV-2. Cell Research, 2021.
43. Zhou, B., et al., SARS-CoV-2 spike D614G change enhances replication and transmission. Nature, 2021. 592(7852): p. 122-127.
44. Hou, Y. J., et al., SARS-CoV-2 D614G variant exhibits efficient replication ex vivo and transmission in vivo. Science, 2020. 370(6523): p. 1464-1468.
45. Liu, Z., et al., Identification of SARS-CoV-2 spike mutations that attenuate monoclonal and serum antibody neutralization. Cell Host Microbe, 2021. 29(3): p. 477-488 e4.
46. Garcia-Beltran, W. F., et al., Multiple SARS-CoV-2 variants escape neutralization by vaccine-induced humoral immunity. Cell, 2021.
47. Starr, T. N., et al., Complete map of SARS-CoV-2 RBD mutations that escape the monoclonal antibody LY-CoV555 and its cocktail with LY-CoV016. Cell Rep Med, 2021. 2(4): p. 100255.
48. Hacisuleyman, E., et al., Vaccine Breakthrough Infections with SARS-CoV-2 Variants. N Engl J Med, 2021.
49. Xiang, Y., et al., Versatile and multivalent nanobodies efficiently neutralize SARS-CoV-2. Science, 2020.
50. Stamatatos, L., et al., mRNA vaccination boosts cross-variant neutralizing antibodies elicited by SARS-CoV-2 infection. Science, 2021.

1-21. (canceled)

22. An antibody binding to SARS-CoV-2 spike glycoprotein or an antigen-binding fragment thereof, wherein the antibody comprises a heavy chain variable region (VH) comprising:
a CDR1 having an amino acid sequence shown in any of SEQ ID NOs: 1, 4, 7, 10, 13, 16 and 19;
a CDR2 having an amino acid sequence shown in any of SEQ ID NOs: 2, 5, 8, 11, 14, 17 and 20; and
a CDR3 having an amino acid sequence shown in any of SEQ ID NOs: 3, 6, 9, 12, 15, 18 and 21.

23. The antibody or antigen-binding fragment of claim 22, wherein the VH comprises a CDR1, a CDR2 and a CDR3 respectively having the amino acid sequences shown in SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, or SEQ ID NOs: 19-21.

24. The antibody or antigen-binding fragment of claim 22, wherein the antibody is univalent, bivalent or multivalent.

25. The antibody or antigen-binding fragment of claim 22, wherein the antibody is mono-specific, bi-specific or multi-specific.

26. A nanobody binding to SARS-CoV-2 spike glycoprotein, comprising:
a CDR1 having the sequence shown in any one of SEQ ID NO: 1, 4, 7, 10, 13, 16 and 19;
a CDR2 having the sequence shown in any one of SEQ ID NO: 2, 5, 8, 11, 14, 17 and 20; and
a CDR3 having the sequence shown in any one of SEQ ID NO: 3, 6, 9, 12, 15, 18 and 21.

27. The nanobody of claim 26, wherein the nanobody comprises a CDR1, a CDR2 and a CDR3 respectively having the amino acid sequences shown in SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, or SEQ ID NOs: 19-21.

28. The nanobody of claim 26, wherein the nanobody comprises a heavy chain frame region, and at least a part of the heavy chain frame region comes from at least one of mouse antibody, human antibody, primate antibody and mutant thereof.

29. The nanobody of claim 26, wherein the nanobody has an amino acid sequence shown in any one of SEQ ID NO: 22-28.

30. A nucleic acid molecule encoding the nanobody of claim 26.

31. The nucleic acid molecule of claim 30, wherein the nucleic acid molecule has a nucleotide sequence shown in any one of SEQ ID NO: 29-35.

32. An antibody-drug conjugate comprising the nanobody of claim 26 conjugated to a therapeutic agent, a diagnostic agent or an imaging agent.

33. A pharmaceutical composition comprising the nanobody of claim 26 and a pharmaceutically acceptable carrier, excipient or diluent.

34. A method for preventing, treating or lessening of a disease caused by SARS-CoV-2 infection in a subject, comprising administering to the subject an effective amount of the nanobody of claim 26.

35. A method for inhibiting binding of spike glycoprotein of SARS-CoV-2 to human angiotensin converting enzyme 2 or blocking SARS-CoV-2 infection in a subject, comprising administering to the subject an effective amount of the nanobody of claim 26.

36. A kit for the detection of SARS-CoV-2 spike glycoprotein RBD or SARS-CoV-2 spike glycoprotein or SARS-CoV-2 comprising the nanobody of claim 26.

37. A nucleic acid molecule encoding the antibody or antigen-binding fragment of claim 22.

38. An antibody-drug conjugate comprising the antibody or antigen-binding fragment of claim 22 conjugated to a therapeutic agent, a diagnostic agent or an imaging agent.

39. A pharmaceutical composition comprising the antibody or antigen-binding fragment of claim 22 and a pharmaceutically acceptable carrier, excipient or diluent.

40. A method for preventing, treating or lessening of a disease caused by SARS-CoV-2 infection in a subject, comprising administering to the subject an effective amount of the antibody or antigen-binding fragment of claim 22.

41. A method for inhibiting binding of spike glycoprotein of SARS-CoV-2 to human angiotensin converting enzyme 2 or blocking SARS-CoV-2 infection in a subject, comprising administering to the subject an effective amount of the antibody or antigen-binding fragment of claim 22.
